(19) United States
(12) Patent Application Publication
(10) Pub. No.: US 2025/0268982 A1
(43) Pub. Date: Aug. 28, 2025

(54) COMPOSITIONS AND METHODS FOR TREATMENT AND/OR PROPHYLAXIS OF PROTEINOPATHIES

(71) Applicant: REGAIN THERAPEUTICS SWEDEN AB, Stockholm (SE)

(72) Inventors: Kariem Ezzat Ahmed, Stockholm (SE); Andrea STURCHIO, Imperia (IT); Alberto ESPAY, Cincinnati, OH (US); Tarja MALM, Siilinjarvi (FI); Samir EL ANDALOUSSI, Stockholm (SE); Joel NORDIN, Stockholm (SE)

(73) Assignee: REGAIN THERAPEUTICS SWEDEN AB, Stockholm (SE)

(21) Appl. No.: 18/563,420

(22) PCT Filed: May 26, 2022
(86) PCT No.: PCT/EP2022/064374

§ 371 Date: Nov. 22, 2023

Related U.S. Application Data
(60) Provisional application No. 63/193,258, filed on May 26, 2021.

(51) Int. Cl. A61K 38/17; A61P 25/28

(52) U.S. Cl. CPC A61K 38/1716; A61P 25/28

(57) ABSTRACT

Non-aggregating protein analogues of proteins involved in a proteinopathy, for example Alzheimer's disease, are provided. The protein has a beta-sheet aggregation domain, and the non-aggregating protein analogue has a beta-sheet destabilizing modification in the beta-sheet aggregation domain but substantially retains wild type protein function. The beta-sheet destabilizing modification can be a substitution of a naturally occurring amino acid for a non-naturally occurring amino acid. Methods of treating a proteinopathy using the non-aggregating protein analogues and methods of designing a non-aggregating protein analogue are provided.

### CROSS-REFERENCE TO RELATED APPLICATIONS

This application claims priority to, and the benefit of, U.S. provisional patent application No. 63/193,258 filed 26 May 2021, which is incorporated by reference herein in its entirety.

### TECHNICAL FIELD

Some aspects relate to compositions for preventing aggregation of proteins in proteinopathies. Some aspects relate to compositions for supplying the native function of a protein while limiting and/or preventing aggregation of said protein in vivo. Some aspects relate to compositions and/or methods useful for the treatment and/or prophylaxis of proteinopathies.

### BACKGROUND

Amyloids are fibrillary protein aggregates formed when certain proteins transition from a natively folded conformation to a cross-beta conformation. In this conformation, the protein molecules are arranged in the form of two oppositely stacked beta-sheets that exclude water molecules between them and have interdigitating generally hydrophobic side chains forming a dry steric zipper. This elongated cross-beta conformation constitutes the basic amyloid fibrillary subunit, the protofilament. The cross-beta architecture can be provided by one folded molecule or two separate molecules, and the beta-sheets can stack in parallel, anti-parallel, face-to-face, or face-to-back orientations.

The protofilaments once formed can associate in a number of different ways to produce different superstructural polymorphs associated with various disorders, including flat fibrillary structures with varied numbers of horizontally stacked protofilaments. These flat fibrillary structures can evolve to amyloid crystals or different twisted ribbon structures of single or multiple intertwined protofilaments, which can further evolve into nanotubes.

A number of disorders have been determined to be amyloid-associated diseases or amyloidopathies, which can be more generally referred to as proteinopathies, including Alzheimer's disease (AD), Parkinson's disease, Lewy body disease, Pick's disease, transmissible spongiform encephalopathies (TSEs) caused by prions such as Creutzfeldt-Jakob disease or Kuru, Huntington's disease, type II diabetes, thyroid medullary carcinoma, pulmonary alveolar proteinosis and atrial amyloidosis resulting from the amyloid accumulation of calcitonin, surfactant protein C and atrial natriuretic factor, amyotrophic lateral sclerosis, Down syndrome, multiple system atrophy, neuronal degeneration with brain iron accumulation type I (Hallervorden-Spatz disease), mild cognitive impairment (MCI), cerebral amyloid angiopathy (CAA), and the like. Examples of proteinopathies include tauopathies, synucleinopathies, prionopathies, TDP-43, and the like. Examples of tauopathies include: Pick's disease, progressive supranuclear palsy, corticobasal degeneration, argyrophilic grain disease, globular glial tauopathies, aging-related tau astrogliopathy, chronic traumatic encephalopathy, primary age-related tauopathy (PART) (or tangle dominant dementia), Parkinsonism-Dementia complex of Guam, postencephalitic Parkinsonism, atypical Parkinsonism of Guadeloupe, diffuse neurofilament tangles with calcification, frontotemporal dementia, Parkinsonism linked to Chromosome tauopathy, and the like. Examples of synucleinopathies include: Parkinson's disease with dementia, pure autonomic failure (PAF), multiple systemic atrophy (MSA), and the like. Examples of prionopathies include fatal familial insomnia (FFI), Gerstmann-Straussler-Scheinker (GSS), and the like. Examples of TDP-43 include: frontotemporal lobar degeneration (FTLD) with TDP43 inclusion, FTLD with moto-neurons disease (FTLD-MND), hippocampal sclerosis, amyotrophic lateral sclerosis, frontotemporal dementia, Parkinsonism liked to Chromosome 17 3R, 4R, or 3R+4R tauopathy, or the like.

Exemplary proteins that are known to be involved in proteinopathies include amyloid-beta protein (Alzheimer's disease), alpha-synuclein (Parkinson's disease), islet amyloid polypeptide (IAPP, type II diabetes), tau (a microtubule-associated protein implicated in frontal temporal dementia with Parkinsonism and Pick's disease), p53 (a tumor suppressor transcription factor, implicated in many cancers), huntingtin protein in Huntington's disease, prion protein in Creutzfelt-Jakob disease, and the like. Table 1 below lists exemplary proteinopathies and the proteins known to be implicated or involved in such proteinopathies.

TABLE 1
Known proteinopathies and the corresponding
protein implicated in the proteinopathy.

| | |
|---|---|
| Alzheimer's disease | amyloid-beta |
| Hereditary cerebral hemorrhage with | amyloid-beta |
| amyloidosis | |
| Parkinson's disease | alpha-synuclein |
| Lewy body dementia | alpha-synuclein |
| Parkinson's disease with dementia | alpha-synuclein |
| Multiple system atrophy | alpha-synuclein |
| Pick's disease | tau protein |
| Progressive supranuclear palsy | tau protein |
| Corticobasal degeneration | tau protein |
| Frontotemporal dementia with | tau protein |
| parkinsonism linked to chr 17 | |
| Argyrophyilic grain disease | tau protein |
| Tangle predominant dementia | tau protein |
| Guam Parkinson dementia comples | tau protein |
| Frontotemporal lobar degeneration | tau protein |
| Chronic traumatic encephalopathy | tau protein |
| Ganglioglioma | tau protein |
| Meningioangiomatosis | tau protein |
| Subacute sclerosing panencephalitis | tau protein |
| Lead encephalopathy | tau protein |
| Tuberous sclerosis | tau protein |
| Hallervorden-Spatz disease | tau protein |
| Lipofuscinosis | tau protein |
| Spongiform encephalopathy including | prion protein (PrP) |
| Creutzfelt-Jakob disease | |
| Fatal insomnia | prion protein (PrP) |
| Gerstmann-Strässler-Scheinker | prion protein (PrP) |
| disease | |
| Huntington disease-like 1 | prion protein (PrP) |
| Spongiform encephalopathy with | prion protein (PrP) |
| neuropsychiatric features | |
| New variant Creutzfelt-Jakob disease | prion protein (PrP) |
| Kuru | prion protein (PrP) |
| Hereditary sensory and autonomic | prion protein (PrP) |
| neuropathy | |
| Type 2 diabetes | Fragments of islet amyloid polypeptide |
| Insulinoma | Islet amyloid polypeptide |
| Primary systemic amyloidosis | Immunoglobulin light chain or fragments |
| | thereof |
| Secondary systemic amyloidosis | Fragments of serum amyloid-A |
| Senile systemic amyloidosis or familial | Transthyretin and fragments thereof |
| amyloid polyneuropathy | |
| Hemodialysis-related amyloidosis | Beta2-microglobulin |
| Hereditary cerebral amyloid | Cystain C |
| angiopathy, Icelandic type | |
| Familial amyloidosis, Finnish type | Gelsolin fragments |
| Familial amyloid polyneuropathy | Fragments of apolipoprotein A-1 |
| Medullar carcinoma of the thyroid | Fragments of calcitonin |
| Atrial amyloidosis | Atrial natriuretic factor |
| Hereditary non-neuropathic systemic | Lysozyme or fragments thereof |
| amyloidosis | |
| Hereditary renal amyloidosis | Fibrinogen fragments |
| Amyloidosis in senescence | Apolipoprotein A-Il |
| Huntingtin disease | Huntingtin |
| Familial British dementia | ABri peptide |
| Familial Danish dementia | ADan peptide |
| Light-chain amyloidosis | Fragments of immunoglobulin light chains |
| Heavy-chain amyloidosis, including | Fragments of immunoglobulin heavy |
| renal | chains |
| AA amyloidosis | Full or N-terminal fragments of serum |
| | amyloid A protein (SAA) |
| Senile systemic amyloidosis | Transthyretin (TTR) |
| Familial amyloidotic polyneuropathy | Transthyretin (TTR) |
| Familial amyloid cardiomyopathy | Transthyretin (TTR) |
| Leptomeningeal amyloidosis | Transthyretin (TTR) |
| Dialysis-related amyloidosis | β₂-microglobulin |
| Hereditary visceral amyloidosis | β₂-microglobulin |
| ApoAI amyloidosis, including in | N-terminal fragments of apolipoprotein A-I |
| various organs | (ApoAI) |
| ApoAII amyloidosis, including renal | C-terminal extended apolipoprotein A-Il |
| | (ApoAII) |
| ApoAIV amyloidosis, including in | N-terminal fragments of apolipoprotein A- |
| various organs | IV (ApoAIV) |
| ApoCII amyloidosis, including renal | Apolipoprotein C-II (ApoCII) |
| ApoCIII amyloidosis, including renal | Apolipoprotein C-III (ApoCIII) |
| Lysozyme amyloidosis, including | Lysozyme |
| visceral | |
| Fibrinogen amyloidosis, including | Fragments of fibrinogen alpha-chain |
| renal | |
| Hereditary cerebral hemorrhage with | N-terminal truncated cystatin C |
| amyloidosis, Icelandic type | |
| Pituitary prolactinoma | N-terminal fragments of prolactin |
| Aortic medial amyloidosis | Medin |
| Gelatinous drop-like corneal dystrophy | Lactotransferrin (lactoferrin) |
| Calcifying epithelial odontogenic | Odontogenic ameloblast-associated |
| tumors | protein (ODAM) |
| Pulmonary alveolar proteinosis | Pulmonary surfactant-associated protein |
| | C (SP-C) |
| Renal amyloidosis | Leukocyte cell-derived chemotaxin-2 |
| | (LECT-2) |
| Lichen amyloidosis | Galectin 7 (Gal-7) |
| Macular amyloidosis | Galectin 7 (Gal-7) |
| Hypotrichosis simplex of the scalp | Corneodesmosin (CDSN) |
| Lattice corneal dystrophy, type 1 | C-terminal fragments of kerato-epithelin |
| Lattice corneal dystrophy, type 3A | C-terminal fragments of kerato-epithelin |
| Lattice corneal dystrophy, Avellino | C-terminal fragments of kerato-epithelin |
| type | |
| Seminal vesicle amyloidosis | Semenogelin-1 (SGI) |
| Prostate cancer | Proteins S100A8/A9 |
| Injection-localized amyloidosis | Enfuvirtide |
| Cerebral autosomal dominant | Neurogenic locus notch homolog protein |
| arteriopathy with subcortical infarcts | 3 (Notch 3) ectodomain |
| and leukoencephalopathy (CADASIL) | |
| Heavy-chain deposition disease, | Immunoglobulin heavy chains |
| including renal | |
| Light-chain deposition disease, | Immunoglobulin light chains |
| including renal | |
| Myeloma cast nephropathy, renal | Immunoglobulin light chains |
| Fanconi syndrome, renal | Immunoglobulin light chains |
| FN glomerulopathy | Fibronectin (FN) |
| Frontotemporal lobar degeneration | TAR DNA-binding protein 43 (TDP-43) |
| with ubiquitin-positive inclusions | |
| Amyotrophic lateral sclerosis | TAR DNA-binding protein 43 (TDP-43) |
| Frontotemporal lobar degeneration | RNA-binding protein FUS (FUS) |
| with ubiquitin-positive inclusions | |
| Amyotrophic lateral sclerosis | RNA-binding protein FUS (FUS) |
| Amyotrophic lateral sclerosis | [Cu-Zn] superoxide dismutase (SOD1) |
| Amyotrophic lateral sclerosis | C9ORF72 protein |
| C1q nephropathy | Complement C1q subcomponent |
| IgA nephropathy (Berger disease) | Immunoglobulin A (IgA) |
| Henoch-Schönlein purpura | Immunoglobulin A (IgA) |
| Primary hyperoxaluria type 1 | Alanine:glyoxylate aminotransferase |
| | (AGT) |
| Multiple myeloma/plasmacytoma | Immunoglobulin G (lgG) |
| (Russell bodies) | |
| Medullary cystic kidney disease 2 | Uromodulin, or Tamm-Horsfall urinary |
| | glycoprotein (THP) |
| Familial juvenile hyperuricemic | Uromodulin, or Tamm-Horsfall urinary |
| nephropathy | glycoprotein (THP) |
| Glomerulocystic kidney disease | Uromodulin, or Tamm-Horsfall urinary |
| | glycoprotein (THP) |
| Spinocerebellar ataxia 1 | Ataxin-1 |
| Sickle cell anemia | Hemoglobin |
| Heinz body anemia | Hemoglobin |
| Inclusion body β-thalassemia | Hemoglobin |
| α1-antitrypsin deficiency | α1-antitrypsin |
| Heredeitary hyperferritinemia cataract | Ferritin light chain |
| syndrome | |
| Alzheimer's disease | Actin |
| Frontotemporal dementia | Actin |
| Cancer | Cellular tumor antigen p53 |

Amyloid-beta peptide is a 39-43 amino acid peptide that is derived by proteolysis from amyloid precursor protein (APP). Amyloid precursor protein (APP) is cleaved by a β-secretase to produce a 99-residue transmembrane fragment C99, which then undergoes further cleavages by γ-secretase to generate the amyloid-beta peptide. The predominant circulating form of amyloid-beta protein is Aβ-40, although Aβ-42 and Aβ-43 are also found in amyloid-beta plaques. The function of amyloid-beta protein is not well understood, but it does appear to play a role in normal synaptic plasticity and memory. A growing body of literature demonstrates the role of Aβ peptides in memory and synaptic plasticity via alpha-7 nicotinic acetylcholine receptor signaling.

Considering Alzheimer's disease as an example proteinopathy, there are numerous different types and causes of Alzheimer's disease. Alzheimer's disease can include Alzheimer's disease arising from any cause or of any other type, including familial Alzheimer's disease (also called autosomal dominant Alzheimer's disease), sporadic Alzheimer's disease, or early-onset sporadic Alzheimer's disease. Apart from being a condition on its own right, mild cognitive impairment may be regarded as an early stage of Alzheimer's disease. Thus, the emergence of mild cognitive impairment may signal the need for initiating therapy to avoid progression to the more profound cognitive impairment typical for Alzheimer's disease. In other words, subjects with mild cognitive impairment are at increased risk of Alzheimer's disease.

A subset of familial Alzheimer's disease cases are caused by specific genetic defects in the presenilin 1 (PSEN1), presenilin 2 (PSEN2) or amyloid-β protein precursor (APP) genes. PSEN1 functions as the catalytic subunit of γ-secretase while mutations in PSEN2 may increase γ-secretase activity. It has been established by numerous studies that the level of soluble Aβ42 in various types of familial Alzheimer's disease, including that caused by mutations in PSEN1, PSEN2 or APP, begins to decline many years before disease onset. Thus, a decrease in soluble levels of Aβ42 is associated with Alzheimer's disease, and occurs long before the disease develops.

A subset of familial Alzheimer's disease is caused by mutations in the Aβ-42 peptide, particularly at residues 19-24 of the peptide. Further, it is believed that the YEVHHQ domain at residues 10-15 of the Aβ42 peptide is also important for native function of the peptide.

In addition to Alzheimer's disease, a decrease in soluble Aβ42 peptide has also been identified in other disorders, including Amyotrophic lateral sclerosis (ALS), frontotemporal dementia, Parkinson's disease, Parkinson's disease dementia, progressive supranuclear palsy, corticobasal degeneration, dementia with Lewy bodies, multiple system atrophy, and neurogenerative dementias. See e.g. Millenhauer et al., J. Neurochem. (2016) 139 (Suppl. 1), 290-317, which is incorporated by reference herein in its entirety.

Alpha-synuclein is a protein that is abundant in the brain, and which is found mainly at the tips of neurons in presynaptic terminals. The function of alpha-synuclein is not well understood, but it is believed to play a role in synaptic vesicle recycling and it may also help to regulate the release of dopamine. Patients with Parkinson's disease or Lewy body dementia generally develop Lewy bodies, which are aggregations of alpha-synuclein, in their brains.

Prion protein is a cellular glycoprotein that has been implicated in Creutzfeldt-Jakob disease. Soluble prion protein in its native conformation is believed to be involved in myelin maintenance and cellular proliferation processes.

Conventional wisdom suggests that many of the disease states caused by proteinopathies are caused by a toxic gain of function of the proteins involved as they aggregate. Most conventional therapies are aimed at minimizing protein aggregation or removing the aggregated proteins (e.g. by reducing protein expression, or by techniques such as immunotherapy aimed at clearing aggregated proteins from cells). However, such therapies have not been particularly successful, and in some cases halting expression of the protein involved in the proteinopathy (for example by knocking out or knocking down the relevant protein) can actually result in a disease phenotype even in the absence of the relevant protein and its aggregated forms. Further, for example in Alzheimer's disease, it has been shown that there is not always a correlation between plaque load and disease severity.

There is emerging evidence that the disease states caused by proteinopathies may in fact be caused by the loss of function of the native proteins that become aggregated in such diseases, particularly in earlier stages of disease. Most amyloid-forming proteins are known or suspected to perform various functions in their native folded state. As such proteins unfold into a cross-beta conformation and form fibrils, the native function of the protein may be lost. Such loss of function of the protein can have deleterious effects on cells that are at least part of the disease process.

There is a general desire for improved therapeutics for proteinopathies. There is a desire for improved therapeutics for disorders involving proteinopathies that can address loss-of-function of the native protein.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Some aspects provide a non-aggregating peptide analogue of the Aβ42 peptide. The Aβ42 peptide has an N-terminal domain corresponding to positions 1-28 of SEQ ID NO: 1, and a beta-sheet aggregation domain corresponding to positions 29-42 of SEQ ID NO: 1. The non-aggregating peptide analogue has a beta-sheet destabilizing modification in the beta sheet aggregation domain, and substantially retains the native function of the Aβ42 peptide except for being non-aggregating. In some aspects, the non-aggregating peptide analogue is for use in the treatment and/or prophylaxis of Alzheimer's disease or mild cognitive impairment or another disorder associated with a decreased level of soluble Aβ42 peptide. In some aspects, a method of screening a subject to determine if the subject is a candidate for the treatment and/or prophylaxis of Alzheimer's disease or another disorder associated with a decreased level of soluble Aβ42 peptide using a non-aggregating peptide analogue as disclosed herein is provided. The concentration of Aβ42 in a sample of cerebrospinal fluid of the subject is determined, and if the measured concentration of Aβ42 in the cerebrospinal fluid sample is less than about 500 pM, preferably less than about 400 pM, preferably less than about 300 pM, more preferably less than 200 pM, the subject is identified as a candidate for treatment and/or prophylaxis via the administration of a non-aggregating peptide analogue as described herein. In some aspects, the method further comprises administering the non-aggregating peptide analogue to the subject.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1, having panels 1.1, 1.2, 1.3, 1.4, 1.5 and 1.6, shows exemplary engineered proteins having a beta-sheet destabilizing mutation in the beta-sheet aggregation domain of the amyloid-beta protein. The presence of a blank shaded block in the table indicates a deletion mutation.

FIG. 2 shows an example embodiment of a method for designing a non-aggregating analogue of a protein implicated in a proteinopathy.

FIG. 3 shows an example embodiment of an engineered protein that is a non-aggregating analogue of a protein implicated in a proteinopathy.

FIG. 4A shows the restoration of phenotype in the Alzheimer's disease animal model 5XFAD demonstrating that administration of an engineered peptide intravenously restored wild type behaviour when administered for five weeks. FIGS. 4B and 4C show parallel results from additional experiments.

FIG. 5A shows the relative plaque load in the cortices of vehicle treated (left side) 5XFAD mice as compared with 5XFAD mice treated for five weeks with an engineered peptide that is a non-aggregating analogue of Aβ42. FIG. 5B shows the corresponding level of plaque accumulation in the hippocampi of the mice.

FIG. 6A shows the aggregation propensity of Aβ42 peptide and two non-aggregating analogues thereof, as measured using a thioflavin-T kinetics assay. FIG. 6B shows the aggregation propensity of Aβ42 peptide and two additional exemplary non-aggregating analogues thereof incorporating a non-naturally occurring amino acid substitution.

FIG. 7A and FIG. 7B show results of assays on SH-SY5Y neuronal cell-line expressing amyloid precursor protein gene with the Swedish mutations. FIG. 7A shows cell proliferation relative to untreated cells, while FIG. 7B shows cell proliferation relative to wild type cells. FIGS. 7C and 7D show data from additional similar experiments showing cell proliferation over untreated cells plotted along a shorter γ-axis to better show comparisons.

FIG. 8 shows results of assays on SH-SY5Y neuronal cells that were treated with 10 μM of γ-secretase inhibitor.

FIGS. 9A and 9B show the levels of soluble Aβ42 that differentiate healthy individuals (cognitively normal or CN) from those with mild cognitive impairment (MCI) or Alzheimer's disease (AD).

FIG. 10 shows adjusted prediction of CDR non-progression with baseline CSF Aβ42 levels.

FIG. 11A-11D shows the percentage of facilitation of α7 nicotinic acetylcholine receptors in cortical neurons in comparison with pre- and post-control responses for an example non-aggregating peptide analogue.

FIGS. 12A-12D and 13 show the percentage of facilitation of α7 nicotinic acetylcholine receptors in cortical neurons in comparison with pre- and post-control responses for additional example non-aggregating peptide analogues.

FIG. 14 shows the percentage facilitation of α7 nicotinic acetylcholine receptors in cortical neurons in comparison with pre- and post-control responses for Aβ42.

FIGS. 15A-15D show the percentage of facilitation of α7 nicotinic acetylcholine receptors in cortical neurons in comparison with pre- and post-control responses for a control scrambled peptide.

FIG. 16 shows that RT88 induces downstream ERK1/2 phosphorylation at pM concentrations.

FIG. 17 shows docking simulations showing that an exemplary non-aggregating peptide analogue of Aβ42 binds to the α7 nicotinic acetylcholine receptor via its N-terminal domain.

FIG. 18 shows a model of the formation of intermolecular beta-sheets, with the position occupied by the methionine at position 35 circled.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The inventors have now determined that administration of engineered peptides/proteins that provide the beneficial wild type function of a peptide/protein implicated in a proteinopathy but which have been modified to avoid or minimize the formation of a cross-beta sheet structure can be therapeutic and/or prophylactic for the proteinopathy. Without being bound by theory, the administration of such proteins is believed to provide equivalent or similar function to the native protein, to address the loss of function of such protein that is believed to be caused by the proteinopathy, without contributing further to aggregation of the protein.

As used herein, the term “engineered peptide” may be used interchangeably with the term “engineered protein”, it being understood by those skilled in the art that both peptides and proteins are made up of amino acids joined by peptide bonds, with proteins being longer chains of amino acids than peptides. The protein involved in a proteinopathy may sometimes be a peptide, and so a reference to protein involved in a proteinopathy also encompasses a peptide involved in a proteinopathy.

As used herein, amino acid sequences for peptides are provided, including a numbering of the positions of each amino acid residue within that sequence, for example positions 1-42 of the Aβ42 peptide (SEQ ID NO: 1). It will be recognized by those skilled in the art that for corresponding peptide analogues, if one or more amino acid residues are deleted, the notional numbering of the positions of the peptide will change, for example if the first residue of the Aβ42 peptide is deleted in a particular peptide analogue, that particular analogue will have only residues 1-41. As used herein, the term “corresponding” when referring to the numbering of the position of an amino acid within a peptide sequence refers to the numbering of the consensus sequence of the peptide as provided herein, and so refers to the corresponding numbering of the amino acid residue relative to the reference sequence when the two peptide sequences are aligned. Thus, the particular analogue in which the first residue of the Aβ42 peptide mentioned above will contain amino acid residues corresponding to residues 2-42 if the Aβ42 peptide as described herein.

As used herein, the term “beta-sheet aggregation domain” refers to a domain of a peptide or protein that is involved in the formation of a cross-beta conformation of the protein that is implicated in a proteinopathy. While the beta-sheet aggregation domain may not in healthy subjects be associated with the formation of a beta-sheet structure or with aggregation, in individuals suffering from or developing proteinopathies, the collective intra- and inter-molecular interactions of the beta-sheet aggregation domain result in the formation of cross-beta sheets that contribute to the formation of amyloid deposits of the protein or peptide. For the Aβ42 peptide, the beta-sheet aggregation domain is located at positions corresponding to residues 29-42 of SEQ ID NO: 1.

The terms “treat”, “treating” and “treatment” refer to an approach for obtaining desired clinical results. Desired clinical results can include, but are not limited to, reduction or alleviation of at least one symptom of a disease. For example, treatment can be diminishment of at least one symptom of disease, diminishment of extent of disease, stabilization of disease state, prevention of spread of disease, delay or slowing of disease progression, palliation of disease, diminishment of disease reoccurrence, remission of disease, prolonging survival with disease, or complete eradication of disease. The term “prophylaxis” includes an approach for preventing disease from occurring or developing from an early stage to a later stage. The term “prevention” in the present context refers to preventive measures resulting in any degree of reduction in the likelihood of developing the condition to be prevented, including a minor, substantial or major reduction in likelihood of developing the condition as well as total prevention. Preferably, the degree of likelihood reduction is at least a minor reduction.

As used herein, the term proteinopathy is used to refer to a disease or disorder caused by the aggregation of a protein or peptide into a cross-beta sheet structure that facilitates the formation of fibrillary aggregates of the protein or peptide. Examples of proteinopathies include amyloidopathies, synucleopathies, tauopathies, and the like.

As used herein, the term “naturally occurring amino acid residue” includes not only the wild type amino acid residue typically found at a particular position within a protein or peptide, but also a mutant amino acid that may occur at that particular position in particular disorders or in an individual subject with a genetic mutation that causes a difference in amino acid sequence at that particular position. An “amino acid analogue” is any non-naturally occurring analogue of a naturally occurring amino acid residue, whether produced synthetically or in vivo via post-translational modification. A non-naturally occurring amino acid analogue may be an analogue that comprises an amino acid. Alternatively, an amino acid analogue may also be devoid of an amino acid moiety, i.e. the non-naturally occurring analogue may comprise any chemical moiety that can be incorporated into an engineered peptide as long as the engineered peptide maintains its native function with the exception of being non-aggregating.

As used herein, amino acid residues are represented with their one-letter code as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine(S), threonine (T), tryptophan (W), tyrosine (Y), valine (V). Unless further specified, all references to amino acid residues herein are to the L-amino acids normally produced by eukaryotes. In some alternative embodiments, the corresponding D-amino acids could be used in place of some or all of the corresponding L-amino acids, including within the beta-sheet aggregation domain and/or as the beta-sheet destabilizing mutation. In alternative embodiments, other non-naturally occurring amino acids or amino acid analogues could be used, including e.g. N-alkyl analogues of the amino acids. Peptides that contain D-amino acids or other non-naturally occurring amino acid residues may have a longer half-life in vivo than peptides that contain only naturally occurring amino acids, for example as a result of serum protein binding and/or enhanced stability against proteases encountered in vivo.

As used herein, the term “conservative amino acid substitution” means an amino acid sequence that differs from a reference sequence by one or more conservative substitutions of one or more amino acid residues relative to a reference molecule, regardless of where such amino acid substitution occurs (i.e. regardless of whether such substitution is within the beta-sheet aggregation domain described herein or outside such domain). Amino acid substitutions that are considered to be “conservative” include any of:
Interchanging any one of A, V, L, or I, or an amino acid analogue substantially similar thereto;
Interchanging any one of S, C, U, T, or M, or an amino acid analogue substantially similar thereto;
Interchanging any one of F, Y, or W, or an amino acid analogue substantially similar thereto;
Interchanging any one of D or E or an amino acid analogue substantially similar thereto;
Interchanging any one of N or Q or an amino acid analogue substantially similar thereto; and/or
Interchanging any one of H, K or R or an amino acid analogue substantially similar thereto.

In some embodiments, an engineered protein that is a non-aggregating analogue of a protein implicated in a proteinopathy that can be used as a therapeutic and/or prophylactic agent for the proteinopathy is provided. In some embodiments, the engineered protein includes one or more beta-sheet destabilizing modifications in the beta-sheet aggregation domain. A beta-sheet destabilizing modification is a modification that interferes with and/or disrupts the formation of a beta-sheet structure, thereby interfering with or preventing the formation of the cross-beta sheet structure that can contribute to the proteinopathy. For example, in some embodiments, a beta-sheet destabilizing modification may disrupt one or more hydrogen bonds that are important to forming the stacked beta-sheet structure and/or the cross-beta sheet structure that can contribute to the proteinopathy. In some embodiments, a beta-sheet destabilizing modification may alter a conformation of the beta-sheet aggregation domain (e.g. by kinking the peptide backbone and/or causing interactions with other residues) to interfere with formation of the stacked beta-sheet structure and/or the cross-beta sheet structure that can contribute to the proteinopathy. In some embodiments, the beta-sheet destabilizing modification does not interfere with, or interferes only minimally with, the native function of the protein, so that the engineered protein retains or substantially retains the wild type activity of the protein.

In some embodiments, the engineered non-aggregating analogue of the protein involved in a proteinopathy is considered to be non-aggregating when the beta-sheet destabilizing modification results in at least a 2-fold, at least a 3-fold or at least a 4-fold lower steady state level of aggregation in a thioflavin-T assay as compared with the wild type protein. In some embodiments, the engineered non-aggregating analogue is an analogue of Aβ42 peptide and has at least a 2-fold, at least a 3-fold or at least a 4-fold lower steady state level of aggregation in a thioflavin-T assay as compared with wild-type Aβ42 having the amino acid sequence of SEQ ID NO:1.

Examples of beta-sheet destabilizing modifications include:
Substitution of a naturally occurring amino acid with a charged or hydrophilic amino acid within the beta-sheet aggregation domain (e.g. K, R, H, E, D, S, T, N, Q or C);
Substitution of a naturally occurring amino acid with a proline (P) residue within the beta-sheet aggregation domain;
Substitution of a naturally occurring amino acid with a glycine (G) residue within the beta-sheet aggregation domain;
Substitution of a naturally occurring amino acid with an amino acid analogue that interferes with beta-sheet formation within the beta-sheet aggregation domain, examples of said amino acid analogues that interfere with beta-sheet formation include 3-hydroxyproline, 4-hydroxyproline, selenocysteine, pyroglutamic acid, carboxyglutamic acid, octenyl alanine, pyrrolysine, palmitoyl aspartate, D-amino acids including D-proline, β-amino acids, γ-amino acids, Homo-amino acids, β-Homo-amino acids, α-methyl amino acids, N-methyl amino acids, N-ethyl amino acids, N-alkylated amino acid derivatives, pyruvic acid derivatives, branched-chain amino acid derivatives, nitro amino acid derivatives, halogenated amino acid derivatives, ring-substituted amino acid derivatives, aromatic amino acid derivatives, linear core amino acids, peptoid derivatives, hydroxylated amino acid derivatives, cyclic amino acids, bicyclic amino acids, 3-amino-3-aryl-propionic acids, 3-amino-4-aryl-butyric acids, amino acids with aromatic spacers, alicyclic amino acids, α-phenylglycine derivatives, or the like.
Deletion of one or more naturally occurring amino acid residues within the beta-sheet aggregation domain, optionally including deletion of up to and including the entirety of the beta-sheet aggregation domain; and/or.
Insertions of one or more amino acid residues within the beta-sheet aggregation domain, e.g. insertion of 1, 2, 3, 4 or 5 amino acid residues within the beta-sheet aggregation domain. In some embodiments, one or more of the inserted amino acid residues is a proline (P) residue. In some embodiments, one or more of the inserted amino acid residues is a glycine (G) residue. In some embodiments, one or more of the inserted amino acid residues is a charged and/or hydrophilic amino acid (e.g. K, R, H, E, D, S, T, N, Q or C). In some embodiments, one or more of the inserted amino acid residues is an amino acid analogue that interferes with beta-sheet formation as described above, examples of said amino acid analogues that interfere with beta-sheet formation include 3-hydroxyproline, 4-hydroxyproline, selenocysteine, pyroglutamic acid, carboxyglutamic acid, octenyl alanine, pyrrolysine, palmitoyl aspartate, D-amino acids including D-proline, β-amino acids, γ-amino acids, Homo-amino acids, β-Homo-amino acids, α-methyl amino acids, N-methyl amino acids, N-ethyl amino acids, N-alkylated amino acid derivatives (preferably with 1, 2 or 3 carbons in the alkyl moiety), pyruvic acid derivatives, branched-chain amino acid derivatives, nitro amino acid derivatives, halogenated amino acid derivatives, ring-substituted amino acid derivatives, aromatic amino acid derivatives, linear core amino acids, peptoid derivatives, hydroxylated amino acid derivatives, cyclic amino acids, bicyclic amino acids, 3-amino-3-aryl-propionic acids, 3-amino-4-aryl-butyric acids, amino acids with aromatic spacers, alicyclic amino acids, α-phenylglycine derivatives, or the like.

Without being bound by theory, it is believed that substitution of such amino acid residues or deletion or insertion of at least one amino acid residue within the beta-sheet aggregation domain of the protein involved in a proteinopathy interferes with the effective formation of a cross-beta sheet structure, thereby limiting or preventing aggregation of the protein while leaving the protein soluble and available to perform its wild-type function. Thus, the engineered protein can be used to restore the loss-of-function defects that may be associated with the proteinopathy, while not aggravating or increasing aggregation of the protein involved in the proteinopathy. To achieve this, in some embodiments, the engineered protein incorporating one or more beta-sheet destabilizing modifications within a beta-sheet aggregation domain fully or substantially retains its native function, e.g. the engineered protein is able to perform its native function at least 50% of the level of the native soluble protein (including e.g. at least 55%, 60%, 65%, 70%, 75%, 80%, 85%, 86%, 87%, 88%, 89%, 90%, 91%, 92%, 93%, 94%, 95%, 96%, 97%, 98%, 99% or 100% of the level of the native soluble protein).

In some embodiments, the non-aggregating peptide analogue is an analogue of Aβ42, and the native function of the Aβ42 is to enhance or induce α7 nicotinic acetylcholine receptor (α7nAChR) mediated Ca²⁺ influx. Typically, the α7nAChR mediated Ca²⁺ influx can be triggered using an agonist (e.g. a small molecule agonist such as PNU 282987), but the influx is increased in the presence of an enhancing molecule such as the non-aggregating peptide analogue of the Aβ42 peptide described herein. The peptide described herein can also induce the influx on its own. In some embodiments, enhancing α7nAChR mediated Ca²⁺ influx means that a level of the α7nAChR mediated Ca²⁺ influx in the presence of the peptide is at least 130%, at least 150%, or at least 170% of a pre-control level of Ca²⁺ influx. In some such embodiments, the non-aggregating peptide analogue is able to perform the native function of enhancing α7nAChR mediated Ca²⁺ influx at a level of at least 50% of the level of the native soluble Aβ42 peptide (including e.g. at least 55%, 60%, 65%, 70%, 75%, 80%, 85%, 86%, 87%, 88%, 89%, 90%, 91%, 92%, 93%, 94%, 95%, 96%, 97%, 98%, 99% or 100% of the level of the native soluble Aβ42 peptide). In some embodiments, the non-aggregating analogue of Aβ42 is able to enhance α7nAChR-mediated Ca²⁺ influx when present in cortical neurons at a concentration of ≤3000 pM, including e.g. ≤300 pM, including e.g. ≤30 pM, including e.g. ≤3 pM. In some embodiments, the non-aggregating analogue of Aβ42 is able to enhance α7nAChR-mediated Ca²⁺ influx when present in neuronal cell-lines such as N2a and SH-SY5Y cells at a concentration of ≤3000 pM, including e.g. ≤300 pM, including e.g. ≤30 pM, including e.g. ≤3 pM. In some embodiments, the non-aggregating peptide analogue of Aβ42 is able to enhance α7nAChR mediated Ca²⁺ influx to a greater extent than wild-type Aβ42 peptide having the amino acid sequence of SEQ ID NO:1, e.g. The α7nAChR mediated Ca²⁺ influx can also be indirectly detected or quantitated by measuring a downstream event, such as ERK1/2 phosphorylation, cell proliferation or neurogenesis. ERK1/2 phosphorylation occurs within minutes of α7nAChR mediated Ca²⁺ influx and can be measured using ELISA. Yet another alternative way of detecting or quantitating native function is to assess rescue of neuronal cells after pharmacological depletion of Aβ42 using γ-secretase inhibitor. Still another alternative way of detecting or quantitating native function is to assess proliferation of neuronal cells which express a non-functional mutant amyloid precursor protein (APP).

In some embodiments, portions of the protein other than the beta-sheet aggregation domain may be important for the native function of the protein. For example, in the case of Aβ42 peptide, without being bound by theory, it is believed that the YEVHHQ domain at residues 10-15 of the peptide and the residues at positions 19-24 of the peptide are important for native protein function, for example for proper binding to the α7 nicotinic acetylcholine receptor (α7nAChR). Thus, in some embodiments, the portions of Aβ42 located N-terminal to the beta-sheet aggregation domain, i.e. positions 1-28 of the peptide, are believed to be important for native peptide function. In this description, these residues at or corresponding to positions 1-28 of the Aβ42 peptide are referred to as the “N-terminal domain”.

The N-terminal domain of the non-aggregating peptide analogue may comprise an amino-acid sequence differing from residues 1-28 of SEQ ID NO: 1 by no more than 6, (preferably 5, more preferably 4, even more preferably 3, yet more preferably 2, still more preferably 1, most preferably 0) deletions, insertions and/or substitutions, preferably conservative substitutions. In some embodiments, the engineered peptide that is a non-aggregating analogue of Aβ42 retains the wild type amino acid sequence of the N-terminal domain, or contains only conservative amino acid substitutions within the N-terminal domain. In some embodiments, the peptide that is a non-aggregating analogue of Aβ42 has zero or at most one, at most two, at most three, at most four, at most five, at most six, at most seven, at most eight, or at most nine conservative amino acid substitutions in the N-terminal domain. The beta-sheet aggregation domain of the non-aggregating analogue comprises an amino-acid sequence differing from positions corresponding to positions residues 29-42 of SEQ ID NO: 1 by no more than 6, (preferably 5, more preferably 4, even more preferably 3, yet more preferably 2, still more preferably 1, most preferably 0) deletions, insertions and/or substitutions, preferably conservative substitutions. Preferably, the non-aggregating peptide analogue as defined comprises, in addition to the beta-sheet destabilizing modification, at most three (preferably two, more preferably one) conservative amino acid substitutions in the beta-sheet aggregation domain relative to SEQ ID NO:1.

In some embodiments, the disease or disorder that is the proteinopathy is Alzheimer's disease, Parkinson's disease, Lewy body disease or Lewy body dementia, Pick's disease, Creutzfeld-Jakob disease, Huntington's disease, amyotrophic lateral sclerosis (ALS), Down syndrome, neuronal degeneration with brain iron accumulation type I (Hallervorde-Spatz disease), Kuru or other transmissible spongiform encephalopathy (TSE), mild cognitive impairment (MCI), cerebral amyloid angiopathy (CAA), vascular dementias, any neurodegenerative disease characterized by abnormal amyloid deposition, any other disease or disorder listed in Table 1, or the like.

In some embodiments, the engineered protein is a non-aggregating analogue of a protein implicated in a proteinopathy, including without limitation amyloid precursor protein (APP), amyloid beta protein (including Aβ-39, Aβ-40, Aβ-42 or Aβ-43 isoforms), alpha-synuclein, prion protein, Huntingtin protein, p53, any protein or peptide listed in Table 1, or the like.

In some embodiments, a nucleic acid encoding the engineered peptide is provided. The nucleic acid can be e.g. DNA or RNA (such as mRNA) that can be transcribed and/or translated by cellular machinery to produce the desired engineered protein in vivo. Any suitable method of genetic engineering (e.g. transfection of cells obtained from and then reintroduced into the body, CRISPR-Cas gene editing, introduction of a suitable expression vector into target cells, or the like) and/or nucleic acid delivery system (e.g. lipid nanoparticles, which can be used to deliver CRISPR-Cas gene editing systems, mRNA or expression vectors to target cells) can be used to supply the nucleic acid encoding the engineered protein to the desired target cells. Any methods now known or developed in future for causing desired cells to express a desired protein could be used in various embodiments to cause cells to express the desired non-aggregating engineered peptide. In some embodiments, including embodiments in which the engineered peptide incorporates a non-naturally occurring amino acid analogue, the engineered peptide is chemically synthesized.

In some embodiments, therapeutic compositions comprising engineered peptides that are non-aggregating analogues or nucleic acids encoding such peptides as described herein are administered in any suitable manner now known or developed in future, including direct administration, genetic engineering techniques, liposome-mediated delivery including lipid nanoparticle delivery, or the like. Modes of direct administration can include subcutaneous, intravenous, intracerebroventricular, intracerebral, intrathecal, intraperitoneal, intramuscular or intravenous injection, infusion, or topical, nasal, oral (including sublingual or buccal), rectal, ocular or otic, or other form of delivery, including pumping or direct injection into the brain of a subject. Modes of liposome-mediated delivery can include direct delivery of the engineered peptide or a nucleic acid (e.g. mRNA) encoding the engineered peptide for expression by a cell, or DNA encoding the engineered peptide together with suitable mechanisms (e.g. CRISPR-Cas gene editing systems) to integrate the DNA into the genome of the cell to facilitate expression of the engineered protein by the cell, or any other mechanism of using a DNA vector as an expression module for the desired peptide.

In some embodiments, the amount of engineered peptide to be administered or caused to be expressed can be determined by the person skilled in the art dependent on the condition to be treated and the mode of administration. In some embodiments, the interval of administration of the engineered peptide can be determined by the person skilled in the art dependent on the condition to be treated and the mode of administration. In some embodiments in which the engineered peptide is a non-aggregating analogue of Aβ42, the amount of engineered peptide to be administered or caused to be expressed is sufficient to provide a concentration of the engineered peptide in the cerebrospinal fluid of a subject of between about 200 and 600 μg/mL, including any value or subrange therebetween, e.g. 250, 300, 350, 400, 450, 500 or 550 μg/mL. The concentration of Aβ42 and/or the engineered peptide in the cerebrospinal fluid of the subject may be determined using liquid chromatography-tandem mass spectrometry or an immunoassay, preferably an ELISA immunoassay (e.g. Elecsys, AlzBio3). The precise target concentration of engineered peptide may vary depending on the method of measurement, the specific engineered peptide and the specific condition, but can be established by routine experimentation involving samples from diseased/at risk individuals compared to heathy controls (see Example 4).

In some embodiments, a method of screening a subject to determine whether the subject is a candidate for treatment and/or prophylaxis of a proteinopathy using an engineered peptide that is a non-aggregating analogue of a protein involved in the proteinopathy is provided. In some embodiments, the proteinopathy is Alzheimer's disease and the protein involved in the proteinopathy is the Aβ42 peptide, or the proteinopathy is another disease or disorder associated with a decreased level of soluble Aβ42 peptide. In some embodiments, the method of screening the subject to determine whether the subject is a candidate for treatment and/or prophylaxis of Alzheimer's disease involves determining a concentration of Aβ42 in the cerebrospinal fluid of the subject. If the measured concentration of Aβ42 in the cerebrospinal fluid is less than about 500 pM, less than about 400 pM, less than about 300 pM, or less than about 200 pM, including e.g. less than about 175, 150, 125 or 100 pM, then the subject is identified as a candidate for treatment and/or prophylaxis of Alzheimer's disease via the administration of a non-aggregating analogue of the Aβ42 peptide as described herein. In some such embodiments, the concentration of Aβ42 in the cerebrospinal fluid of the subject is determined using liquid chromatography-tandem mass spectrometry or an immunoassay (e.g. Elecsys, AlzBio3).

In some embodiments, the subject has familial Alzheimer's disease, including familial Alzheimer's disease caused by mutations in PSEN1, PSEN2, or AβPP. Without being bound by theory, it is believed that the mutations associated with familial Alzheimer's disease may reduce the level of soluble Aβ42 peptide and/or interfere with the normal function of the peptide. Thus, supplementing the level of functional Aβ42 peptide in the subject via administration of an engineered peptide that is a non-aggregating analogue of Aβ42 as described herein may be particularly beneficial for such subjects.

In some embodiments, the subject is a mammalian subject. In some embodiments, the subject is a human subject.

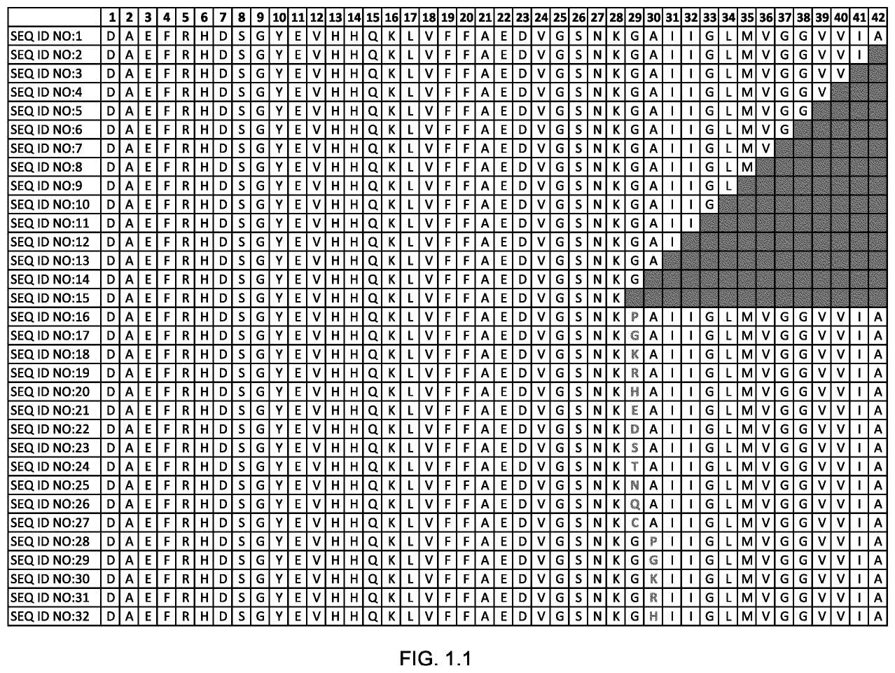

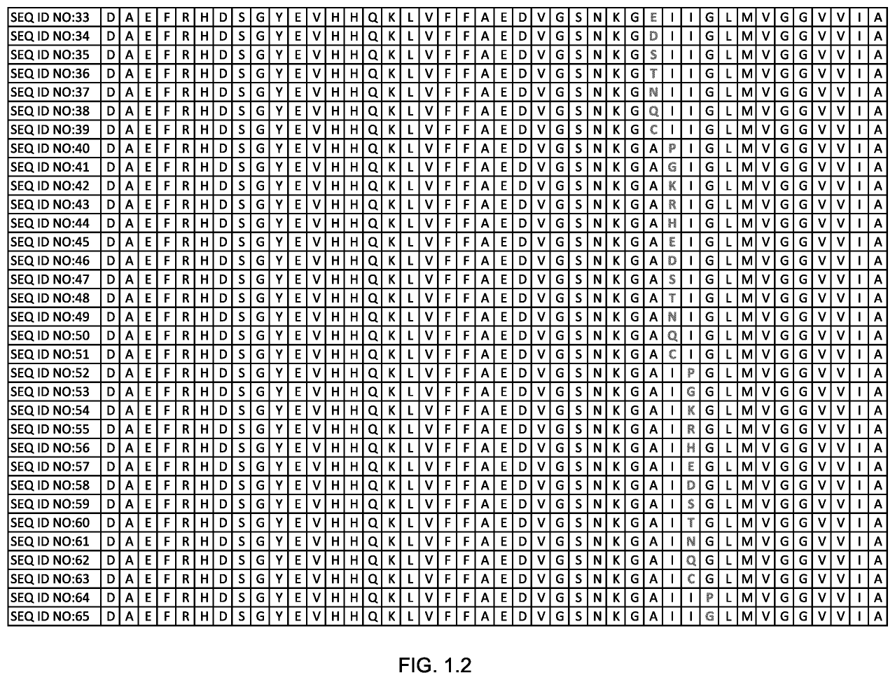

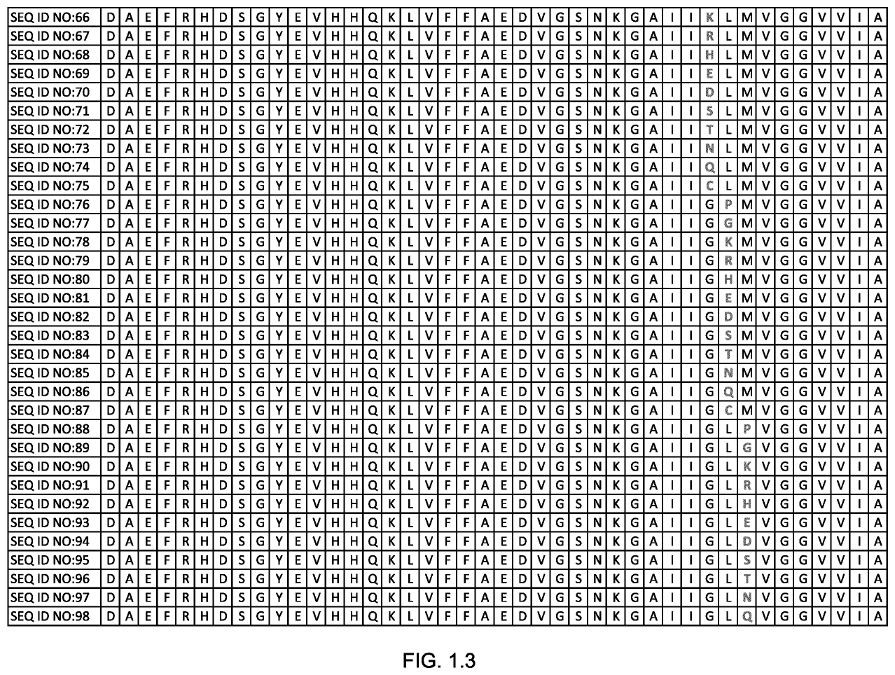

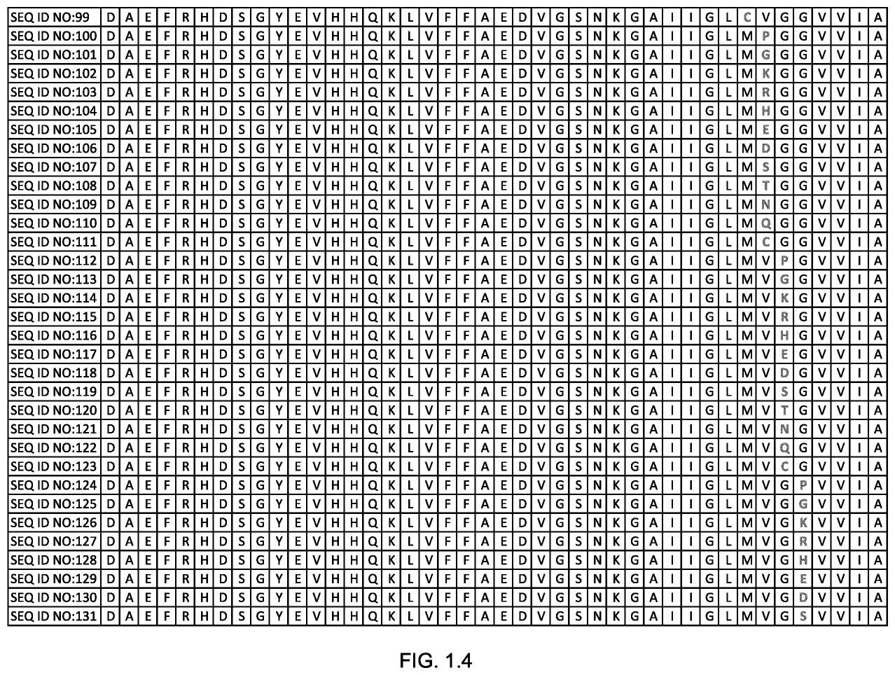

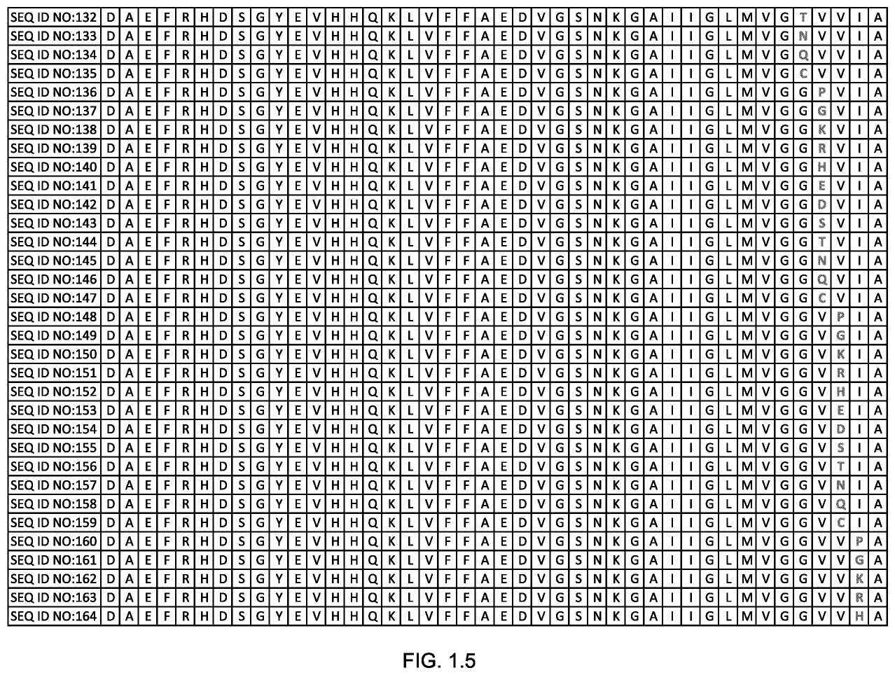

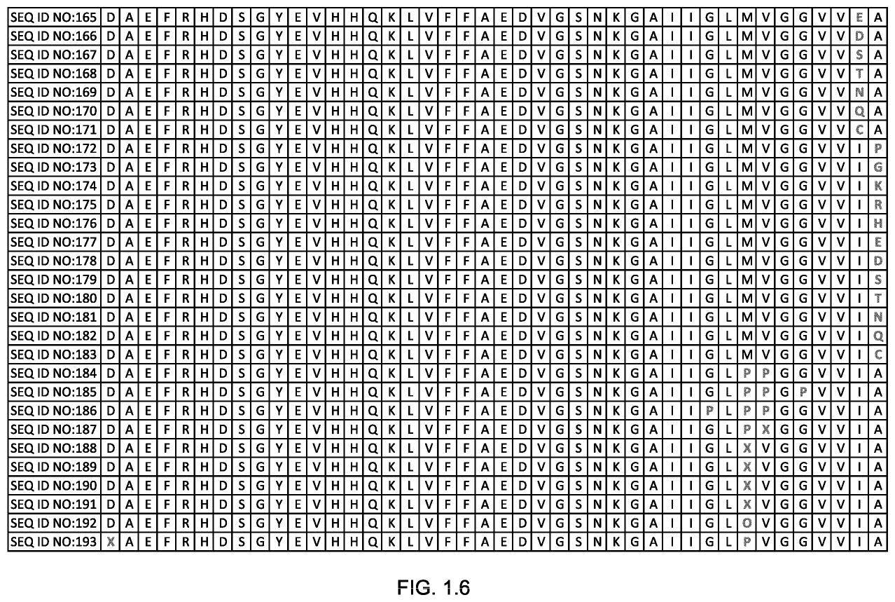

With specific reference to the example of the amyloid-beta peptide implicated in Alzheimer's disease and other disorders, the wild type sequence of human Aβ-42 is as set forth in SEQ ID NO: 1, and this is shown in FIG. 1. The beta-sheet aggregation domain of Aβ-42 is believed to be the carboxy-terminal residues 29-42 thereof. Accordingly, in some embodiments, the engineered peptide is a form of amyloid-beta peptide having a beta-sheet destabilizing modification at one or more of positions 29-42 thereof. The beta-sheet destabilizing modification may be a substitution of one or more of the amino acids at positions 29-42 (including any position therebetween, e.g. positions 29, 30, 31, 32, 33, 34, 35, 36, 37, 38, 39, 40, 41 or 42) with an amino acid that interferes with beta-sheet formation such as P, G, K, R, H, E, D, S, T, N, Q, C or an amino acid analogue that interferes with beta-sheet formation including e.g. 3-hydroxyproline, 4-hydroxyproline, selenocysteine, pyroglutamic acid, carboxyglutamic acid, octenyl alanine, pyrrolysine, palmitoyl aspartate, D-amino acids including D-proline, β-amino acids, γ-amino acids, Homo-amino acids, β-Homo-amino acids, α-methyl amino acids, N-methyl amino acids, N-ethyl amino acids, N-alkylated amino acid derivatives (preferably with 1, 2 or 3 carbons in the alkyl moiety), pyruvic acid derivatives, branched-chain amino acid derivatives, nitro amino acid derivatives, halogenated amino acid derivatives, ring-substituted amino acid derivatives, aromatic amino acid derivatives, linear core amino acids, peptoid derivatives, hydroxylated amino acid derivatives, cyclic amino acids, bicyclic amino acids, 3-amino-3-aryl-propionic acids, 3-amino-4-aryl-butyric acids, amino acids with aromatic spacers, alicyclic amino acids, α-phenylglycine derivatives, or the like. The beta-sheet destabilizing modification may be a deletion of the naturally occurring amino acid residue at that position, or insertion of between 1 and 5 amino acid residues at that position (optionally where at least one of the inserted amino acid residues is P, G, K, R, H, E, D, S, T, N, Q, or C or an amino acid analogue that interferes with beta-sheet formation as set forth above). Examples of proteins having beta-sheet destabilizing modifications include the amino acid sequences of SEQ ID NO: 2-193 shown in FIG. 1 or SEQ ID NOs: 194-207 or SEQ ID NOs: 712-742 (wherein X indicates the position of a beta-sheet destabilizing modification) or any combination thereof. Additional examples include any sequence that is at least 90% identical to any of SEQ ID NO: 2-207, including at least 91%, 92%, 93%, 94%, 95%, 96%, 97%, 98%, 99% or 99.9% identical thereto. In some embodiments, the engineered protein does not include a peptide having the sequence of SEQ ID NO:3, SEQ ID NO:5 or SEQ ID NO: 15. In the illustrated preferred embodiments of FIG. 1, the X at position 36 of SEQ ID NO: 187 represents octenyl alanine; the X at position 35 of SEQ ID NO: 188 represents D-proline, the X at position 35 of SEQ ID NO:189 represents pyroglutamic acid; the X at position 35 of SEQ ID NO: 190 represents N-methyl methionine, the X at position 35 of SEQ ID NO: 191 represents carboxyglutamic acid, the X at position 35 of SEQ ID NO: 192 represents pyrrolysine, and the X at position 1 of SEQ ID NO: 193 represents palmitoyl-aspartate. Peptides analogues according to SEQ ID NO: 190 or 192 are the most preferred.

In some embodiments, the engineered peptide is Aβ-43 having any of the foregoing sequences described for Aβ-42, with an additional T residue provided at the carboxy-terminal end of the peptide to provide the Aβ-43 isoform.

In some embodiments, the engineered peptide is Aβ-40 having any of the foregoing sequences described for Aβ-42, with the two carboxy-terminal amino acids removed to provide the Aβ-40 isoform.

In some embodiments, the engineered peptide is Aβ-39 having any of the foregoing sequences described for Aβ-42, with the three carboxy-terminal amino acids removed to provide the Aβ-39 isoform.

With specific reference to the example of the alpha-synuclein protein implicated in Parkinson's disease, the wild type sequence of human alpha-synuclein is as set forth in SEQ ID NO:208. The beta-sheet aggregation domain of alpha-synuclein is believed to be residues 61-95 thereof. Accordingly, in some embodiments, the engineered protein is a form of alpha-synuclein having a beta-sheet destabilizing modification at one or more of positions 61-95 thereof. In some embodiments, the beta-sheet destabilizing modification is a substitution of one or more of the amino acids at positions 61-95 (including any position therebetween, e.g. positions 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94 or 95) with an amino acid that interferes with beta-sheet formation or with an amino acid analogue that interferes with beta-sheet formation as described above, or by deletion of the naturally occurring amino acid residue at that position, or by insertion of between 1 and 5 amino acid residues at that position (optionally wherein at least one of the inserted amino acid residues is P, G, K, R, H, E, D, S, T, N, Q, or C or an amino acid analogue that interferes with beta-sheet formation), including for example a protein having any of the sequences of SEQ ID NO:209-663 or any of the sequences of SEQ ID NO:664-698 (wherein X indicates the position of a beta-sheet destabilizing modification) or any combination thereof, or any sequence that is at least 90% identical thereto, including at least 91%, 92%, 93%, 94%, 95%, 96%, 97%, 98%, 99% or 99.9% identical thereto.

With specific reference to the example of prion protein implicated in Creutzfeld-Jakob disease, the wild type sequence of human prion protein is as set forth in SEQ ID NO: 699. The beta-sheet aggregation domain of the human prion protein is believed to be residues 109-121 thereof. Accordingly, in some embodiments, the engineered protein is a form of prion protein having a beta-sheet destabilizing modification at one or more of positions 109-121 thereof. In some embodiments, the beta-sheet destabilizing modification is a substitution of one or more of the amino acids at positions 109-121 thereof (including any position therebetween, e.g. positions 109, 110, 111, 112, 113, 114, 115, 116, 117, 118, 119, 120 or 121) with an amino acid that interferes with beta-sheet formation or with an amino acid analogue that interferes with beta-sheet formation as described above, or by deletion of the naturally occurring amino acid residue at that position, or by insertion of between 1 and 5 amino acid residues at that position (optionally wherein at least one of the inserted amino acid residues is P, G, K, R, H, E, D, S, T, N, Q, or C or an amino acid analogue that interferes with beta-sheet formation), including for example a protein having the amino acid sequence of any one of SEQ ID NO:700-710 or any combination thereof, or any sequence that is at least 90% identical thereto, including at least 91%, 92%, 93%, 94%, 95%, 96%, 97%, 98%, 99% or 99.9% identical thereto, wherein the X denotes the position of the beta-sheet destabilizing modification.

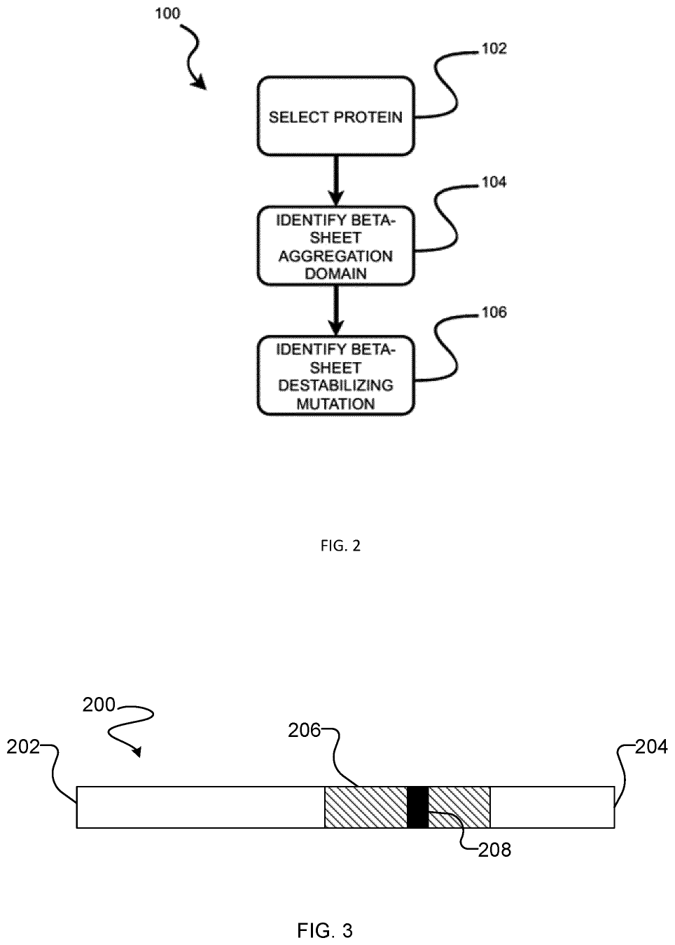

With reference to FIG. 2, in some embodiments a method 100 of designing a non-aggregating analogue of a protein implicated in a proteinopathy is described. At 102, the target protein that is implicated in the proteinopathy is selected. For example, where the proteinopathy is Alzheimer's disease, amyloid-beta could be selected as the target protein (or peptide) that is implicated in the proteinopathy based on available scientific literature. Where the proteinopathy is Parkinson's disease, alpha-synuclein could be selected as the target protein that is implicated in the proteinopathy based on available scientific literature. Where the proteinopathy is Creutzfeld-Jakob disease, prion protein could be selected as the target protein that is implicated in the proteinopathy based on available scientific literature. In other embodiments where the protein that is implicated in the proteinopathy is not known, suitable experiments could be conducted to determine the identity of a protein that has aggregated in a proteinopathy to select the relevant protein for further steps.

At 104, the beta-sheet aggregation domain of the selected protein is identified. For example, structural biology studies (e.g. X-ray crystallography or other studies) or a literature review of such studies can be carried out to evaluate the amyloid structure of the selected protein, to identify the domain(s) involved in aggregation.

At 106, one or more beta-sheet destabilizing modifications that may prevent the formation of a cross-beta sheet structure by the selected protein are identified. The beta-sheet destabilizing modification(s) are made in some embodiments by modifying and/or deleting certain naturally occurring amino acid residues within the beta-sheet aggregation domain. For example, one or more naturally occurring amino acid residues within the beta-sheet aggregation domain may be selected for deletion, or for substitution with G, P, K, R, H, E, D, S, T, N, Q or C or an amino acid analogue that disrupts beta-sheet formation as described above, or the like.

Once the beta-sheet destabilizing modification(s) have been identified by applying method 100, then a suitable construct for administration in vivo (e.g. an engineered peptide or a nucleic acid encoding such a peptide) can be made and administered to a subject suffering from or at risk of developing the proteinopathy as described above.

FIG. 3 shows an example embodiment of an engineered protein 200 that is a non-aggregating analogue of a protein involved in a proteinopathy. Engineered protein 200 has an N-terminus 202, a C-terminus 204, and a beta-sheet aggregation domain 206. Within beta-sheet aggregation domain 206, a beta-sheet destabilizing modification is engineered into the peptide sequence, so that engineered protein 200 will be a non-aggregating analogue of a protein involved in a proteinopathy.

With specific reference to engineered peptides useful for the treatment of Alzheimer's Disease, in some embodiments, one or more of the amino acids at positions 29, 30, 31, 32, 33, 34, 35, 36, 37, 38, 39, 40, 41 or 42 of Aβ42 is substituted with a non-naturally occurring amino acid that disrupts beta-sheet formation. In some embodiments, the non-naturally occurring amino acid that disrupts beta-sheet formation is an N-alkyl analogue of the naturally occurring amino acid, for example an N-methyl analogue or an N-ethyl analogue of the naturally occurring amino acid. Without being bound by theory, it is believed that substitution of an N-alkyl analogue of the naturally occurring amino acid within the beta-sheet aggregation domain can disrupt the formation of intermolecular hydrogen bonds between aggregating peptides, while preserving the amino acid side chain and therefore preserving the normal function of the peptide.

In some specific embodiments, one or more of the following substitutions of a non-naturally occurring amino acid that disrupts beta-sheet formation is made in the Aβ42 peptide to provide an engineered non-aggregating peptide analogue: G29 is N-methyl glycine or N-ethyl glycine, A30 is N-methyl alanine or N-ethyl alanine, I31 is N-methyl isoleucine or N-ethyl isoleucine, I32 is N-methyl isoleucine or N-ethyl isoleucine, G33 is N-methyl glycine or N-ethyl glycine, L34 is N-methyl leucine or N-ethyl leucine, M35 is N-methyl methionine or N-ethyl methionine, V36 is N-methyl valine or N-ethyl valine, G37 is N-methyl glycine or N-ethyl glycine, G38 is N-methyl glycine or N-ethyl glycine, V39 is N-methyl valine or N-ethyl valine, V40 is N-methyl valine or N-ethyl valine, 141 is N-methyl isoleucine or N-ethyl isoleucine, and/or A42 is N-methyl alanine or N-ethyl alanine. In some embodiments, the non-aggregating peptide analogue maintains or substantially maintains the wild type function of Aβ42.

In some specific embodiments, the substitution of a non-naturally occurring amino acid in the engineered non-aggregating analogue of the Aβ42 peptide is made at one or both of positions 35 and 36. Without being bound by theory, it is believed that M35 lies in a strategic position in the cross-β amyloid conformation of Aβ42, so that this residue and adjacent residues may represent particularly desirable target sites for modification with a non-naturally occurring amino acid to help strategically disrupt intermolecular hydrogen bonding, thereby decreasing the aggregation propensity of the peptide. In some embodiments, the engineered non-aggregating analogue of the Aβ42 peptide has the non-naturally occurring amino acid N-methyl methionine or N-ethyl methionine substituted for M35. In some embodiments, the engineered non-aggregating analogue of the Aβ42 peptide has the non-naturally occurring amino acid N-methyl valine or N-ethyl valine substituted for V36.

### EXAMPLES

Specific embodiments are further described with reference to the following examples, which are intended to be illustrative and not limiting in nature.

### Mouse Study Methods

Female wild type and 5xFAD transgenic mice (Jackson laboratories) were used for the mouse behavioural experiment. Mice were individually housed in controlled humidity, temperature and light conditions, and had ad libitum access to food and water. Randomization to treatment groups was carried out by using GraphPad QuickCalcs (GraphPad Software, San Diego, CA, USA), and all data analysis was performed blinded to the experimental groups. All the animal experiments followed the Council of Europe Legislation and Regulation for Animal Protection and are approved by the National Animal Experiment Board of Finland. Every effort was made to minimize the harm and suffering of the animals. The mice were treated with the vehicle or peptide preparations once a week i.v. starting at the age of 1 month until 3 months of age. Behavioral testing: the 5xFAD mice have deficits in nest building test starting at 3 months of age. Briefly, the soft bedding material and plastic shelter tube are moved to the one end of the cage and soft tissue paper (17 cm×17 cm) is placed in the other end of the cage. After 24 hours, a picture of the cage is taken, and the points given according to the set scale. 0 points are given if tissue paper remains untouched, 5 points are given when animal incorporates tissue into nest. Sacrifice and sample processing: At the time of sacrifice, the mice were anesthetized using 250 mg/kg Avertin® (Sigma-Aldrich, St. Louis, MO, USA) and perfused transcardially with saline containing 2500 IU/L heparin (Heparin LEO 5000 IU/ml, Leo Pharma A/S, Ballerup, Denmark). Terminal blood samples were taken at the time of sacrifice (approximately 300 μl of blood) using 1:10 dilution of 3.8% sodium citrate as anticoagulant. The blood samples were centrifuged first 1500 g for 6 minutes, after which the supernatant was removed to a new tube and further centrifuged for 12 000 g for 3 minutes. The resulting plasma was stored in two separate aliquots at −80 degrees for further analysis. The brains were dissected out and cut mid-sagittally into left and right hemispheres. The left hemispheres were post-fixed in 4% paraformaldehyde (PFA, Sigma-Aldrich, St. Louis, MO, USA) for 20 hours, cryoprotected in 30% sucrose in PB for 2 days and frozen on liquid nitrogen. The cortices and hippocampi of the right hemispheres were freshly frozen.

Immunohistochemistry: The brains were post-fixed in 4% paraformaldehyde (PFA, Sigma-Aldrich, St. Louis, MO, USA) for 20 hours, followed by cryoprotection in 30% sucrose for 48 hours. The brains were frozen on liquid nitrogen and cut coronally into 20 μm thick sections with a cryostat (Leica Microsystems, Wetzlar, Germany) in anti-freeze solution. The immunohistochemical staining for WO2, GFAP and Iba1 was carried out on six consecutive sections at 400 μm intervals. The brain sections were incubated overnight at room temperature with primary antibody (Iba-1 1:250 dilution, Wako Chemicals, Tokyo, Japan). Secondary antibody was applied on sections after three washes in 0.05% Tween®20 (Sigma-Aldrich, St. Louis, MO, USA) in PBS. Fluorescent Alexa 488 or 568-conjugated secondary antibodies (1:200 dilution, Abcam, Cambridge, UK) was used for visualization of the immunoreactivities. For quantification of the immunoreactivities, hippocampal areas were imaged using 4 or 10× magnification on an AX70 microscope (Olympus corporation, Tokyo, Japan) with an adjacent digital camera (Color View 12 or F-View, Soft imaging system, Muenster, Germany) running AnalySis software (Soft Imaging System). Quantification of the immunoreactivities was performed using ImagePro Plus software (Media Cybernetics Inc., Rockville, MD, USA) at a predefined range and presented as relative immunoreactive area. All analyses will be performed blinded to the study groups.

THT method. ThT (Sigma-Aldrich) was prepared at 4 mM in MQ water. 50 μL of 500 μg/ml peptide (in normal saline) were incubated with 250 μL of 2.4 mM ThT solution (in water). ThT fluorescence was measured at 440 nm excitation and 480 nm emission in a black, clear-bottom 96-well plates (Corning, USA) at 10-15 min intervals (from bottom with periodic shaking) over 12-24 h on SpectraMax i3 microplate reader (Molecular Devices, USA). Curves were fitted using GraphPad Prism software.

In-vitro method. WT SH-SY5Y neuroblastoma cell line and SH-SY5Y cells stably expressing amyloid-precursor protein with the Swedish mutations (APP-SH-SY5Y) were grown in in growth medium Dulbecco's modified Eagle's medium (DMEM) with 5% FBS (Fetal Bovine Serum) ThermoFisher, USA), 1% Penicillin/Streptomycin (ThermoFisher, USA) at 37° C. and 5% CO2. The day before treatments, cells were seeded in 96-well cell culture plate in DMEM medium without FBS. On the day of treatment, WT SH-SY5Y were treated with 10 μM γ-secretase inhibitor (aldehyde 2-naphthoyl-VF-CHO) together with peptides at different concentrations for 48 hours. APP-SH-SY5Y were treated only with peptides for 72 hours. Cell proliferation was then measured using the WST-1 assay (Sigma-Aldrich) according to manufacturer's protocol.

Clinical method. To find the minimum required level of Aβ42 required for normal cognition, the inventors studied data from the Alzheimer's Disease Neuroimaging Initiative (ADNI), which is a study of over 2700 participants aged between 55 and 90 years (Weiner et al., Alzheimers Dement. 2013 September; 9 (5): e111-94). The inventors studied the levels of cerebrospinal fluid (CSF) Aβ42 within a sample of ADNI participants that were all positive for amyloid plaques based on positron emission tomography tests. The inventors found that amyloid positive individuals with normal cognition (CN) have an average CSF Aβ42 level of 900 pg/ml, which was significantly (asterisks representing significant differences, **P<0.01, and ***P<0.001) higher than CSF Aβ42 levels in individuals with mild cognitive impairment (MCI) and Alzheimer's disease (AD). The inventors conclude that reaching a CSF concentration of 900 μg/ml or equivalent in a subject's cerebrospinal fluid using active Aβ42 analogues is a reasonable therapeutic target for replacement therapy.

### Example 1—In Vivo Demonstration of Replacement Therapy

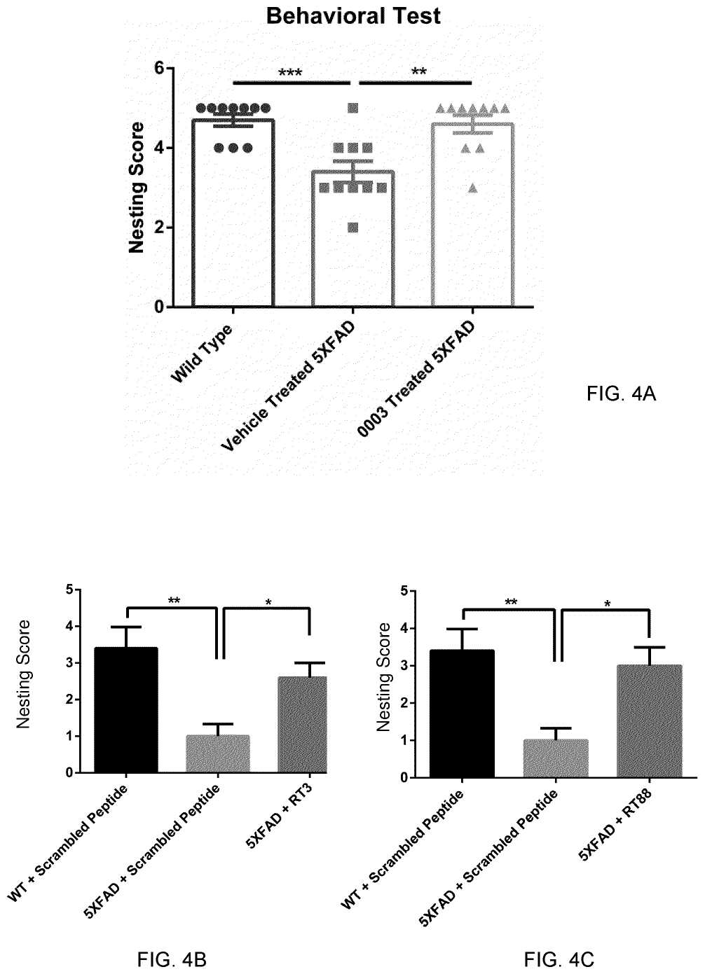

The results shown in FIGS. 4A, 4B and 4C demonstrate that replacement therapy using the engineered version of Aβ42 (Aβ40) having SEQ ID NO:3 with a beta-sheet destabilizing modification by virtue of the deletion of residues 41 and 42 of the beta-sheet aggregation domain leads to phenotype restoration in a well-established AD animal model (5XFAD mice).

Briefly, mice were administered a peptide having the sequence of SEQ ID NO:3 (Aβ-40) intravenously at a dosage of 5 mg/kg/day for five weeks.

As can be seen from the results shown in FIG. 4A, wild type mice exhibited a behavioral score of approximately 5/5, whereas the untreated 5XFAD mice exhibited an average behavioral score of 4/5. Administration of the engineered peptide Aβ-40 that is a non-aggregating analogue of Aβ-42 by virtue of the deletion of residues 41 and 42 of the beta-sheet aggregation domain for a period of five weeks restored mice to a behavioral score of approximately 5/5 (far right). Parallel results for wild type mice administered a scrambled peptide (having the sequence of SEQ ID NO:711) and 5XFAD mice administered the scrambled peptide as controls, together with the phenotype rescue demonstrated by administration of non-aggregating peptides RT3 (having the sequence of SEQ ID NO:3 (Aβ-40)) and RT88 (having the sequence of SEQ ID NO:88) are shown in FIGS. 4B and 4C, respectively.

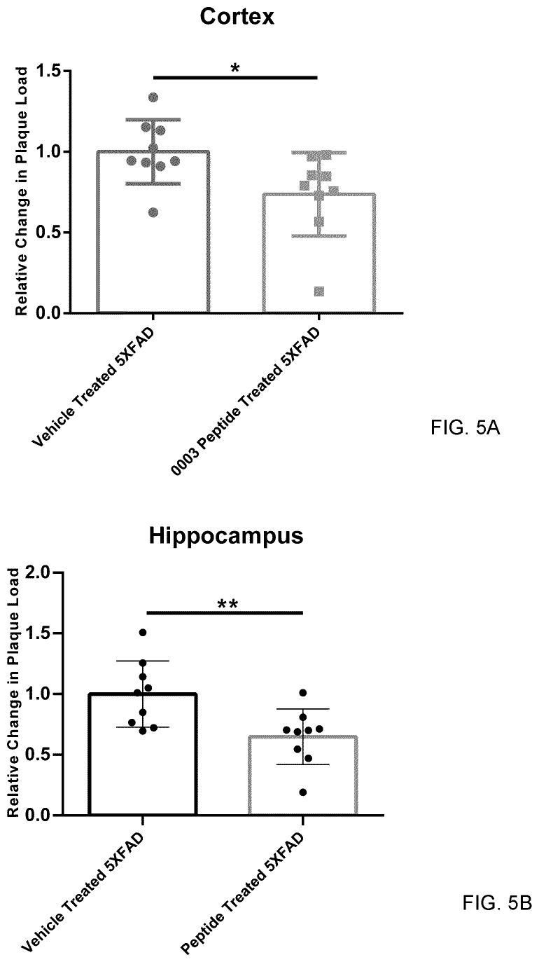

As can be seen from FIG. 5A, 5XFAD mice that were treated with the engineered peptide that is a non-aggregating analogue of Aβ42 (SEQ ID NO:3) also exhibited reduced plaque load in the cortex as compared with vehicle treated animals. FIG. 5B shows a corresponding decreased level of plaque accumulation in the hippocampi of the treated animals as well.

### Example 2—Evaluation of Aggregation Propensity of Aβ42 Analogues

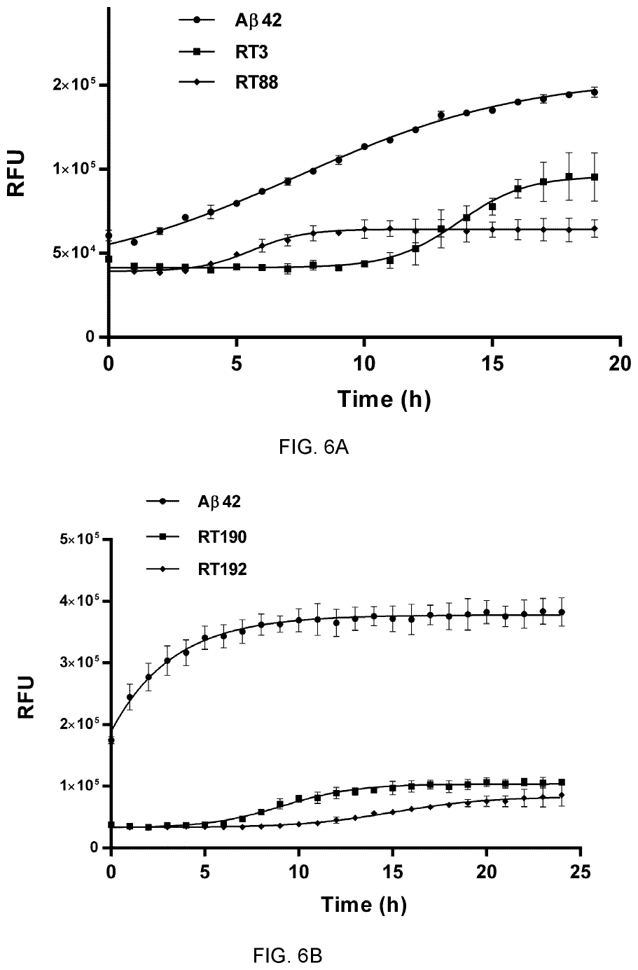

A thioflavin-T kinetics study was carried out on peptides having five different sequences, SEQ ID NO:88 having a proline (P) substitution in place of methionine (M) at position 35, wild type Aβ42 (SEQ ID NO:1), Aβ40 (SEQ ID NO:3), which has the last two C-terminal residues in the beta-sheet aggregation domain of Aβ42 deleted, SEQ ID NO: 190, which has a non-naturally occurring amino acid N-methyl methione substituted for the naturally occurring methionine at position 35, and SEQ ID NO:192, which has a non-naturally occurring amino acid pyrrolysine substituted for the naturally occurring methionine at position 35. As shown in FIG. 6A, the peptide having SEQ ID NO:88 has a lower aggregation propensity than the peptide having SEQ ID NO:3, which has a lower aggregation propensity than wild type Aβ42 having SEQ ID NO:1. As shown in FIG. 6B, the peptides having SEQ ID NO: 190 and SEQ ID NO: 192 also had lower aggregation propensity than wild type Aβ42 (SEQ ID NO:1).

### Example 3—Evaluation of Cell Proliferation in SH-SY5Y Neuronal Cell Line

Experiments were conducted to demonstrate that non-aggregating analogues of Aβ42 can enhance proliferation in neuronal cells which express mutant amyloid precursor protein (APP) and rescue neuronal cells after pharmacological depletion of Aβ42 using γ-secretase inhibitor.

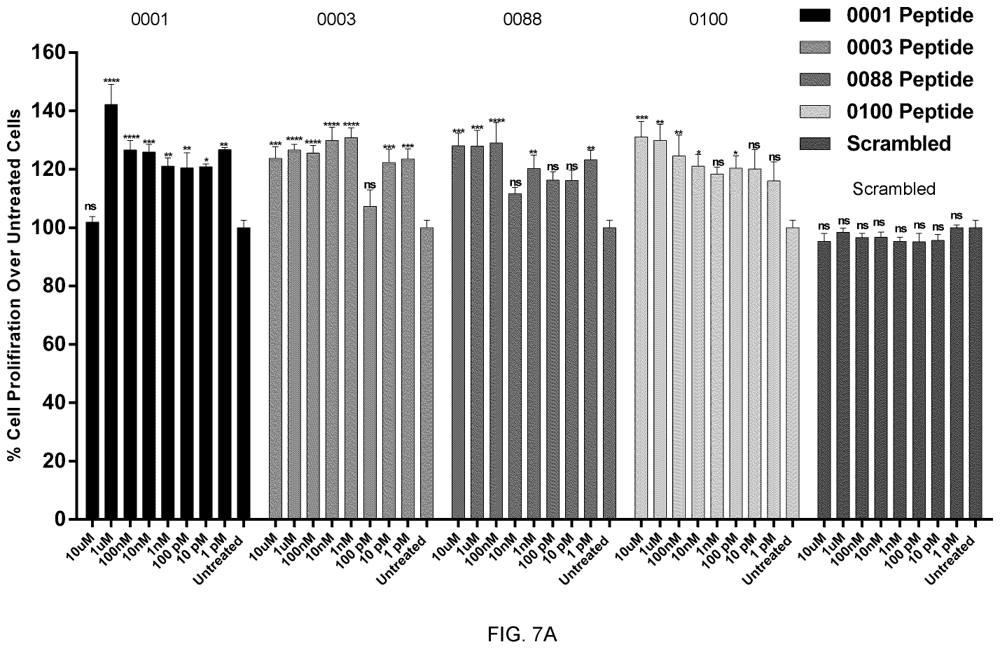

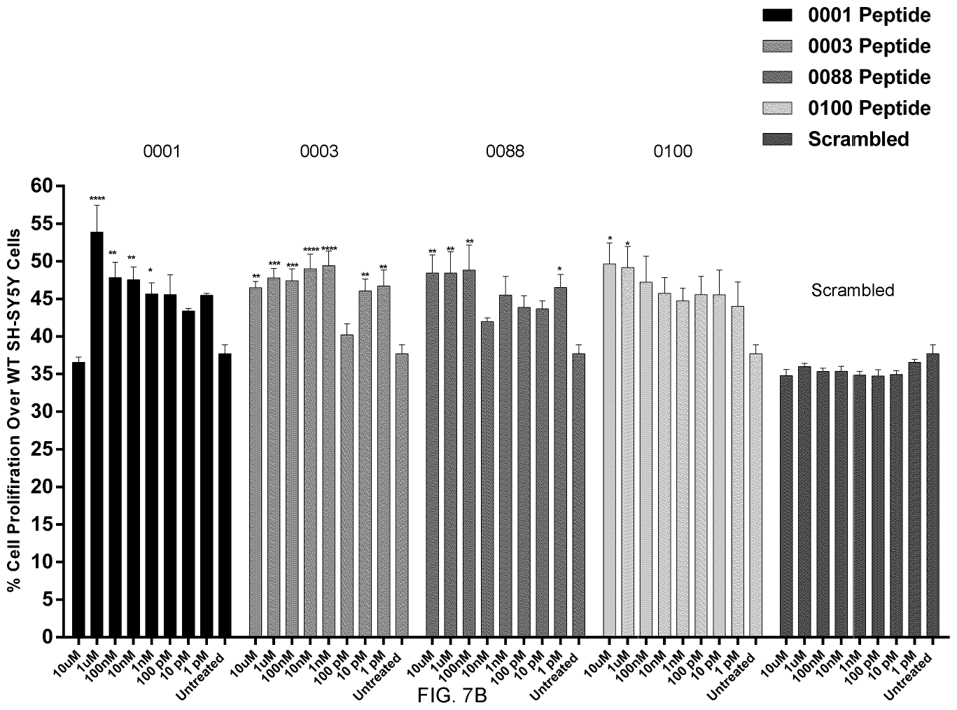

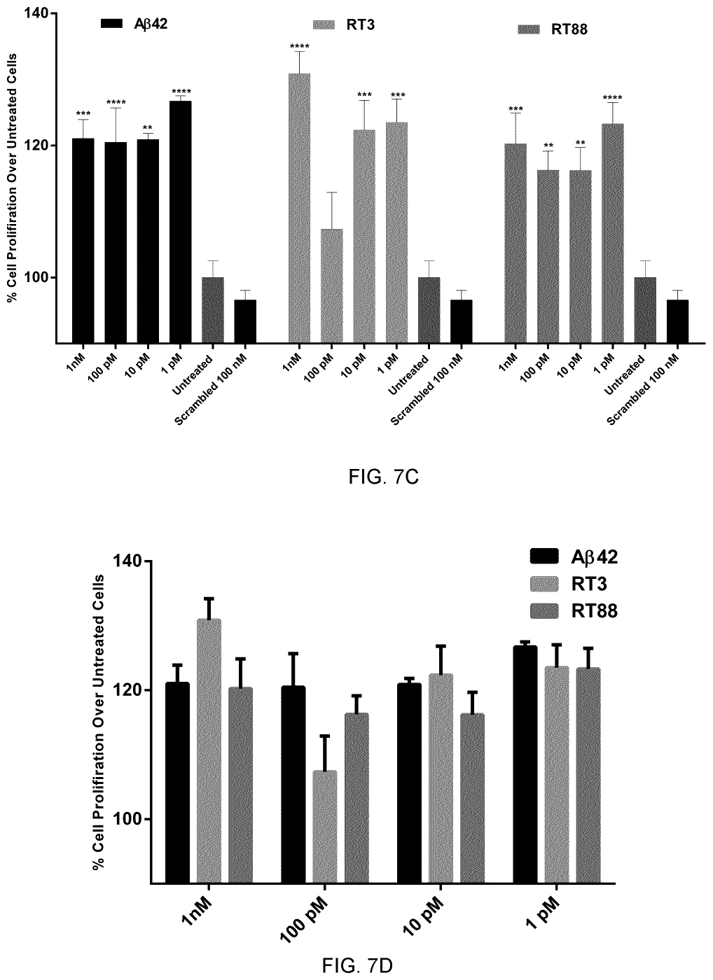

FIG. 7A shows the percentage cell proliferation over untreated cells in assays on SH-SY5Y neuronal cell-line expressing amyloid precursor protein gene with the Swedish mutations (APP-SH-SY5Y). Cells were treated with different concentrations of Aβ42 or non-aggregating analogues thereof or a scrambled control peptide (YHAGVDKEVVFDEGAGAEHGLAQKIVRGFGVSDVSMIHINLF, SEQ ID NO:711) for 72 h in serum-free conditions, then cell proliferation was measured using the WST-1 assay. Results represent the mean of two experiments in quadruplets, with asterisks representing significant differences (significant differences in comparison untreated APP-SH-SY5Y cells were assessed using one-way ANOVA with Dunnetts's multiple comparison test and are indicated by *P<0.05, **P<0.01, and ***P<0.001, ns: non significant.). 0001 Peptide is wild type Aβ42 having SEQ ID NO:1. 0003 Peptide is Aβ40 peptide having SEQ ID NO:3. 0088 Peptide is an Aβ42 analogue having SEQ ID NO:88 (M35P modification), and 0100 Peptide is an Aβ42 analogue having SEQ ID NO: 100 (V36P).

FIG. 7B shows the percentage cell proliferation over wild type cells in assays on SH-SY5Y neuronal cell-line expressing amyloid precursor protein gene with the Swedish mutations (APP-SH-SY5Y). Cells were treated with different concentrations of Aβ42 or non-aggregating analogues thereof or a scrambled control peptide having SEQ ID NO:711 for 72 h in serum-free conditions, then cell proliferation was measured using the WST-1 assay. Results represent the mean of two experiments in quadruplets, with asterisks representing significant differences (significant differences in comparison untreated wild type SH-SY5Y cells were assessed using one-way ANOVA with Dunnett's multiple comparison test and are indicated by *P<0.05, **P<0.01, and ***P<0.001, ns: non significant.). 0001 Peptide is wild type Aβ42 having SEQ ID NO:1. 0003 Peptide is Aβ40 peptide having SEQ ID NO:3. 0088 Peptide is an Aβ42 analogue having SEQ ID NO:88 (M35P modification), and 0100 Peptide is an Aβ42 analogue having SEQ ID NO: 100 (V36P).

FIGS. 7C and 7D show data from additional similar experiments showing cell proliferation over untreated cells plotted along a shorter γ-axis to better show comparisons.

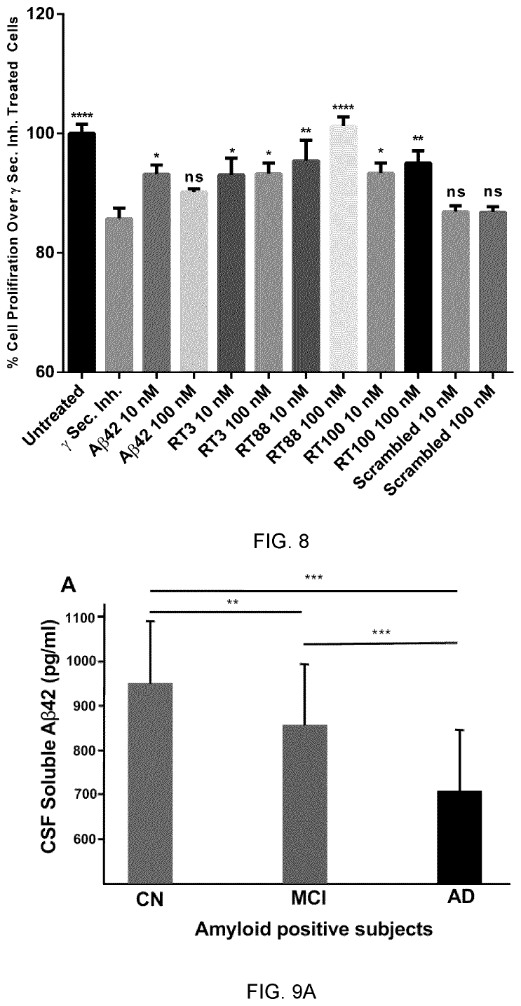

FIG. 8 shows SH-SY5Y neuronal cells that were treated with 10 μM of γ-secretase inhibitor (aldehyde 2-naphthoyl-VF-CHO) then treated with different concentrations of Aβ42 or non-aggregating analogues thereof or a scrambled control peptide peptides (sequences as above) for 48 hours in serum-free conditions, then cell proliferation was measured using the WST-1 assay. Results represent the mean of quadruplet treatments, with asterisks representing significant differences compared to γ-secretase inhibitor treated cells. Significant differences were assessed using one-way ANOVA with Dunnett's multiple comparison test and are indicated by *P<0.05, **P<0.01, and ***P<0.001, ns: non significant.

### Example 4—Determination of Importance of Soluble Aβ42 for Cognitive Function

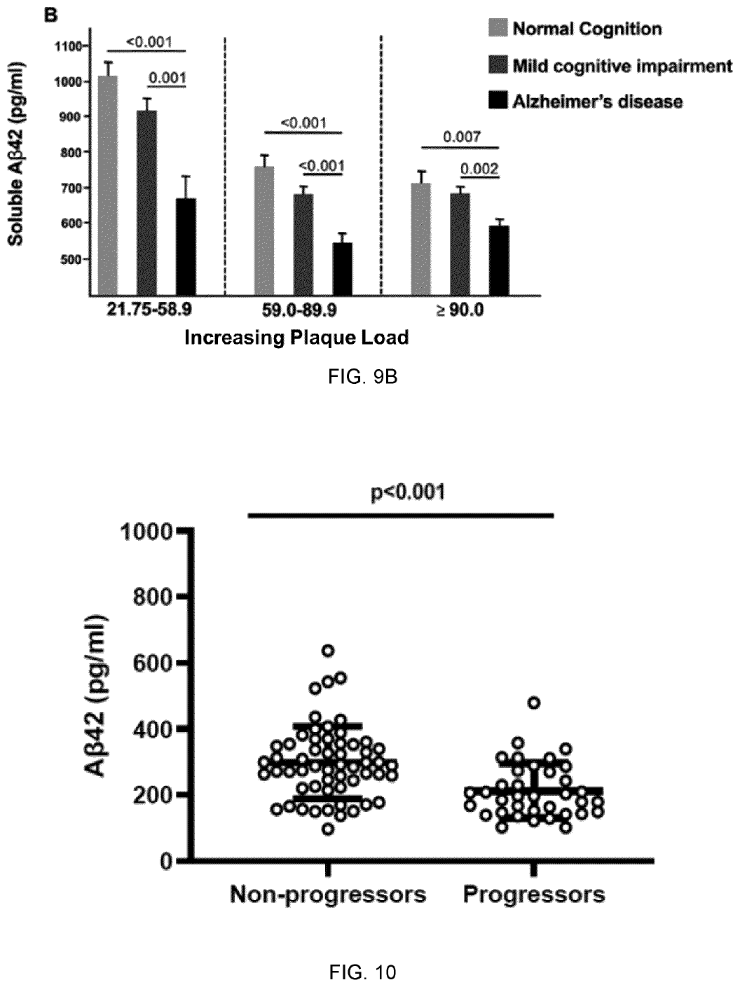

FIG. 9A and FIG. 9B show that decreasing levels of soluble Aβ42 peptide in cerebrospinal fluid is associated with decreasing cognitive function, even across patients with increasing plaque load (FIG. 9B). In this study, levels of Aβ42 in cerebrospinal fluid (CSF) were determined using an ELISA immunoassay (Elecsys) according to data provided by the Alzheimer's Disease Neuroimaging Initiative (ADNI). Soluble Aβ42 across all subjects in each diagnostic category is shown in FIG. 9A, while FIG. 9B shows soluble Aβ42 levels in each diagnostic category across CL tertiles, where CL tertiles were determined by quantifying standard uptake value ratio (SUVR) for a PET scan for amyloidosis, quantifying SUVR across cortical gray matter, normalized by the whole cerebellum and dividing into SUVR tertiles; SUVR levels were converted in centiloids (CL) using the specific equation for each tracer as provided by ADNI. Lower levels of soluble Aβ42 were associated with mild cognitive impairment, with even lower levels associated with the presence of Alzheimer's disease. See also EClinical Medicine (2021) Vol. 38 100988, the entirety of which is incorporated by reference herein. Thus, decreasing levels of soluble Aβ42 are associated with decreasing levels of cognitive function.

FIG. 10 shows a comparison of cerebrospinal fluid Aβ42 levels between non-progressors and progressors to a clinical dementia rating (CDR) in a PiB-PET positive cohort (i.e. patients exhibiting amyloid plaques) in a retrospective longitudinal study among mutation carriers participating in the Dominantly Inherited Alzheimer Network (DIAN) cohort study. In this study, CSF levels of Aβ42 were evaluated using the AlzBio3 assay from Fujirebio, Malvern, PA. Non-CDR progressors had a higher value of Aβ42 in CSF (297.73±13.66) vs. CDR progressors (218.73±17.22); overall cohort: non-CDR progressors (380.83±14.5) vs. CDR progressors (313.35±26.46). Error bar represents the standard error of mean. CDR progression was defined as any increase in CDR over the follow-up period of the study. Patients were defined as amyloid PiB-PET-positive if their standard uptake (SUVR) of Pittsburgh compound B PET (PIB-PET) was greater than or equal to 1.42. Different assays may produce different absolute numbers for the level of soluble Aβ42 in CSF, but the overall trend of lower levels of soluble Aβ42 being associated with poorer outcomes is consistent.

Replacement therapy in various embodiments will aim to restore the levels of soluble functional protein to this level using non aggregating analogues.

### Example 5—Effect of Non-Aggregating Peptide Analogues on the α7 Nicotinic Acetylcholine Receptors in Cortical Neurons

Live cell calcium imaging of cultured WT mouse cortical neurons with PC-driven fast application of combination of the α7 agonist PNU 282987 (1 μM) and positive allosteric modulator (PAM) of α7 PAM PNU 120596 (10 μM) was carried out. This combination uncovers the full activity range of α7nAChR. The specificity of α7 mediated effects was validated with α7nAChR blockers MLA and α-BungTX. As discussed below, it was determined that treatment with a non-aggregating analogue of Aβ42 enhances α7 nAChR mediated Ca⁺² influx, showing that co-application of the non-aggregating analogs of Aβ42 at pM concentration promotes/facilitates/enhances the ability of PAM PNU 120596 to activate α7 receptor, which suggests that the non-aggregating analogue of Aβ42 functions as a positive modulators of the receptor activity. The middle bar in each figure (PNU4) represents the percentage of enhancement in Ca⁺² influx mediated by the non-aggregating peptide analogue compared to the baseline signal before (PNU3) and after (PNU5) application.

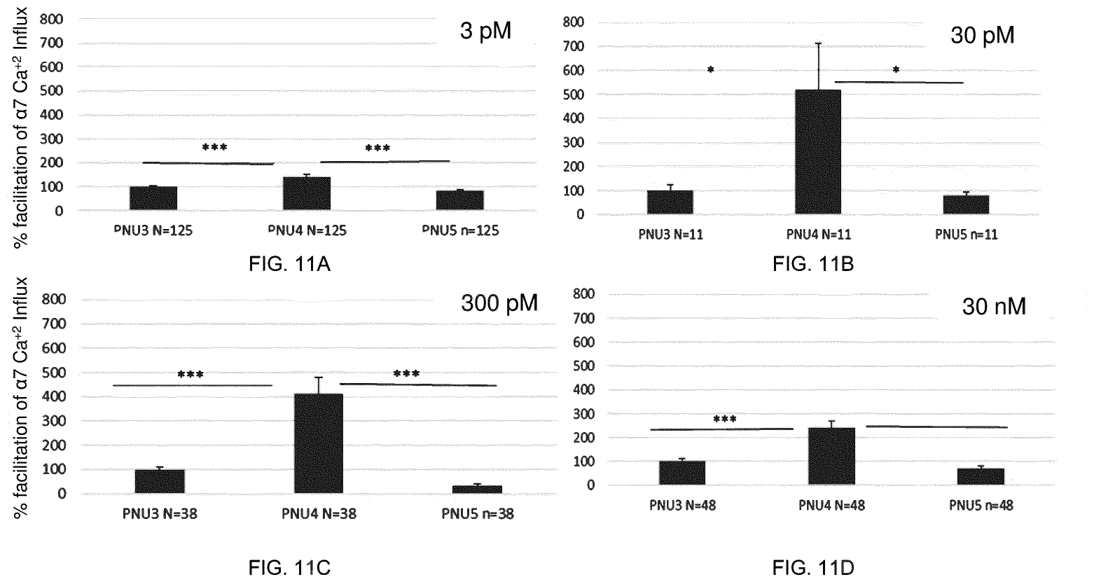

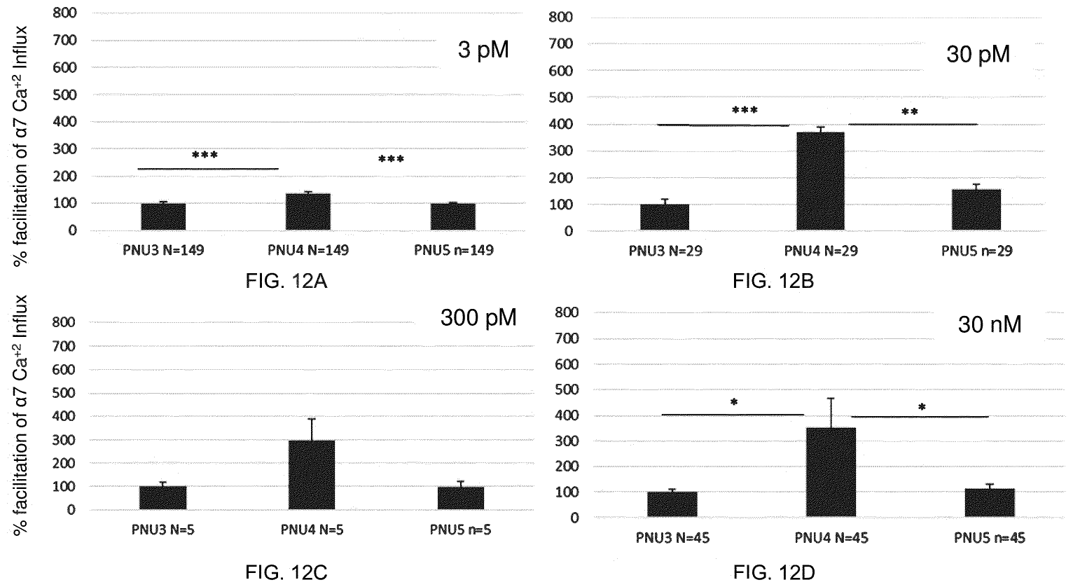

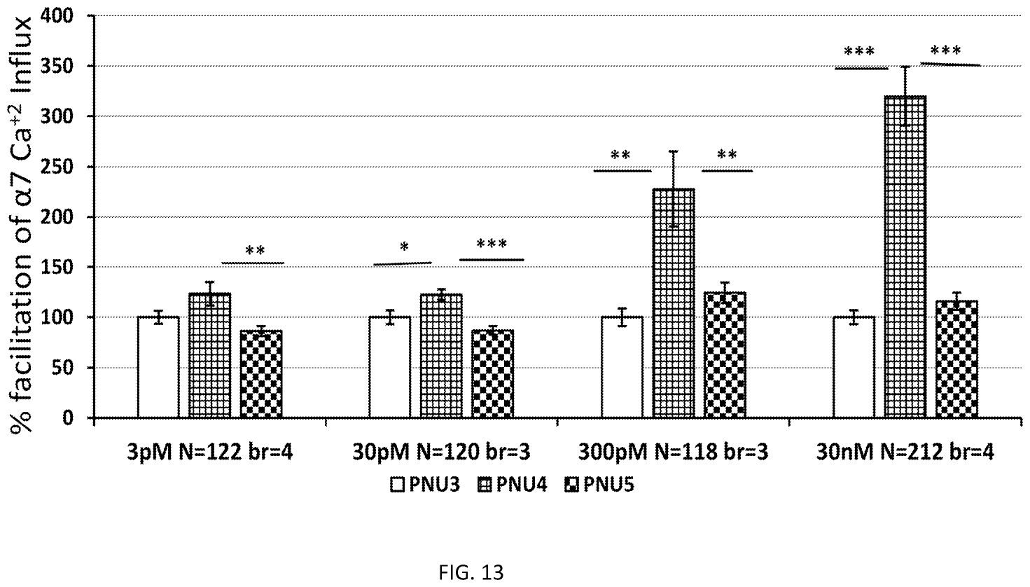

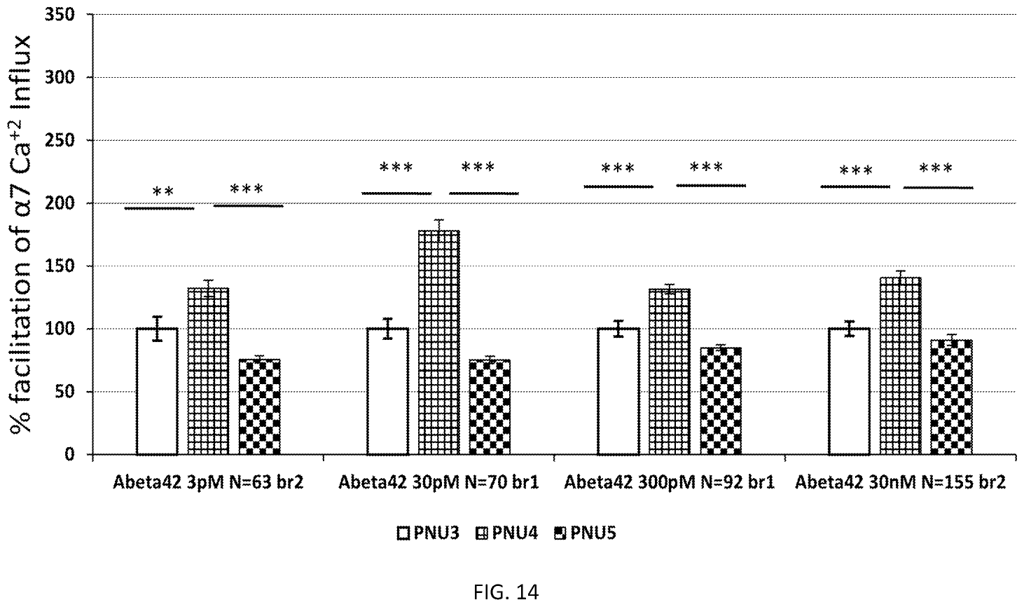

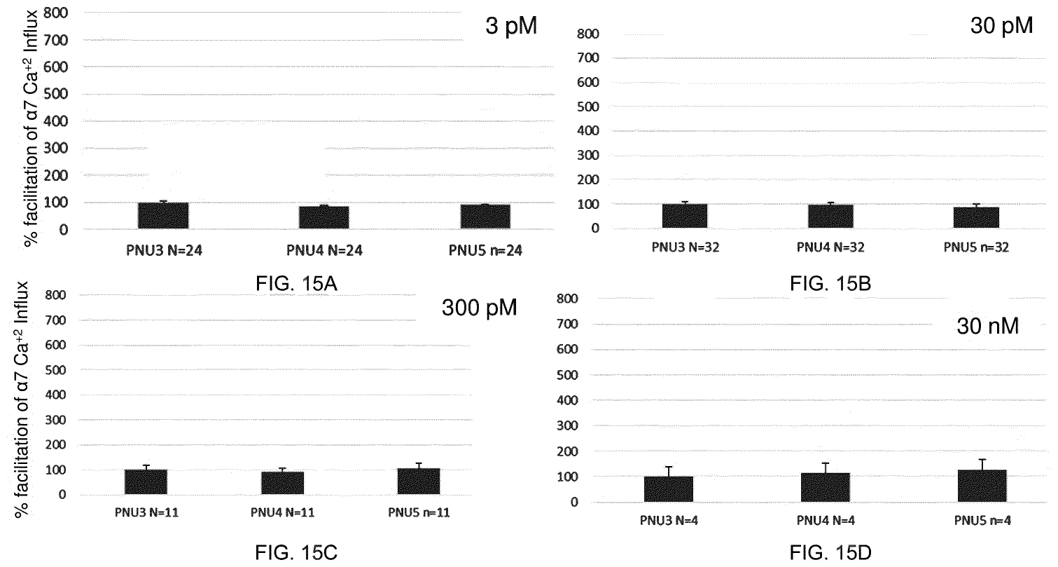

FIGS. 11A-11D show the percentage of facilitation of α7 nicotinic acetylcholine receptors in cortical neurons in comparison with pre- and post-control responses for an example non-aggregating peptide analogue having the amino acid sequence of SEQ ID NO: 88 at the indicated concentrations. FIGS. 12A-12D and 13 show the percentage of facilitation of α7 nicotinic acetylcholine receptors in cortical neurons in comparison with pre- and post-control responses for additional example non-aggregating peptide analogues having the amino acid sequences of SEQ ID NO:3 and SEQ ID NO: 190, respectively, at the indicated concentrations. FIG. 14 shows the percentage facilitation of α7 nicotinic acetylcholine receptors in cortical neurons in comparison with pre- and post-control responses for Aβ42 having SEQ ID NO:1, and FIGS. 15A-15D shows the percentage of facilitation of α7 nicotinic acetylcholine receptors in cortical neurons in comparison with pre- and post-control responses for a control scrambled peptide having the amino acid sequence of SEQ ID NO:711.

These results show that co-application of non-aggregating analogues at pM concentration promotes the ability of PAM PNU 120596 to activate α7 receptor, which without being bound by theory suggests that the analogue functions as a positive modulator of the receptor activity.

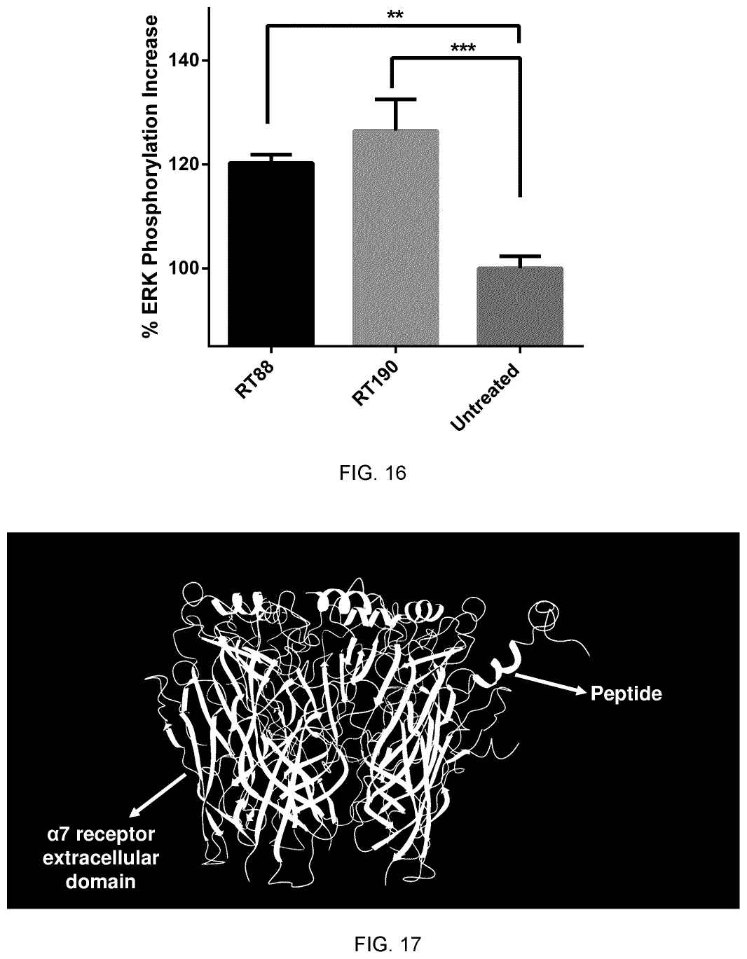

FIG. 16 shows that a non-aggregating peptide analogue having the amino acid sequence of SEQ ID NO:88 or SEQ ID NO: 190 induces downstream ERK1/2 phosphorylation in SHSY-5Y cells after 5 minutes of treatment with the peptide at pM concentrations. Phosphorylation of ERK is important for cell proliferation, neurogenesis and synaptic plasticity, and is downstream of calcium influx through the α7 receptor. To obtain the data shown in FIG. 16, SH-SY5Y cells were treated with 30 pM peptide for 5 minutes, then lysed and ERK phosphorylation quantified via a commercial ELISA kit. The results shown in FIG. 16 demonstrate that the tested non-aggregating analogues of Aβ42 activate the neurogenic signaling pathways mediated by α7 binding and Ca⁺² influx. Although Aβ42 was not used as a control in FIG. 16, parallel experiments comparing the activity of Aβ42 against the non-aggregating peptide analogues showed higher activity on the part of the non-aggregating peptide analogues.

### Example 6—Structural Studies

FIG. 17 shows the results of docking simulations showing that a peptide having the amino acid sequence of SEQ ID NO:88 binds to the α7 nicotinic acetylcholine receptor via the N-terminal domain of the peptide analogue. The peptide is visible on the right-hand side of the figure where the N-terminal portion of the peptide is received within a binding pocket formed within the extracellular domain of the α7 receptor. Without being bound by theory, the fact that binding to the α7 receptor occurs via the N-terminal domain of the peptide analogue, whereas the C-terminal domain is the beta-sheet aggregation domain, is believed to support that making modifications at the C-terminal portion of the peptide is less likely to interfere with the normal biological function of the Aβ42 peptide.

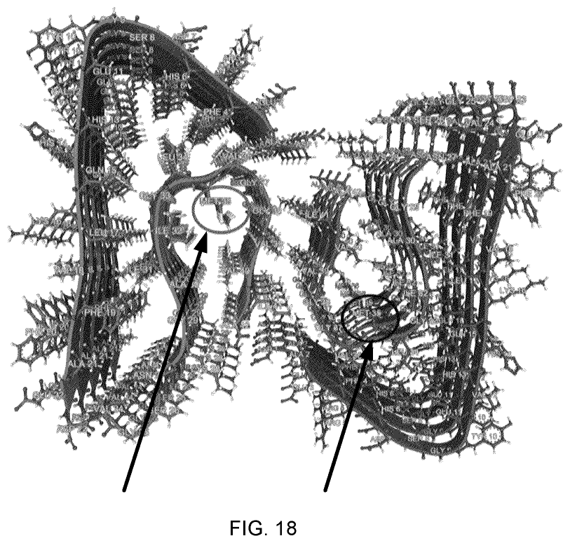

FIG. 18 shows a structural representation of the oppositely stacked beta-sheets of the Aβ42 peptide that are believed to form the protofilaments that form amyloid deposits in Alzheimer's disease. The position occupied by methionine 35 in each of the oppositely stacked beta-sheets is circled and indicated by an arrow to show the position of this residue in stabilizing the cross-β amyloid conformation.

The foregoing examples demonstrate that administration of a non-aggregating analogue of Aβ42 can be used to ameliorate symptoms of Alzheimer's disease in an accepted animal model for the disease. Based on these results, it can be soundly predicted that the use of non-aggregating analogues of other proteins implicated in various proteinopathies can be used in the treatment and/or prophylaxis of such proteinopathies.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### REFERENCES

The following references are of potential interest with respect to the subject matter described herein. Each of the following references is incorporated by reference herein in its entirety.
Kanaan and Manfredsson, Journal of Parkinson's Disease 2:249-267 (2012).
Malmberg et al., Frontiers in Neuroscience, 14:256 (2020).
Wood et al., Biochemistry, 34 (3): 724-30 (1995).
Chiti and Dobson, Annu. Rev. Biochem. 86:26-68 (2017).
US 2008/0063636 to Mori et al.
US 2010/0081613 to Arancio et al.
U.S. Pat. No. 6,689,753 to Soto-Jara
U.S. Pat. No. 7,342,091 to Kapurniotu et al.
WO 03/045128 to Frangione et al.
U.S. Pat. No. 5,686,411 to Gaeta et al.

Certain embodiments are further defined with reference to the following aspects, which are illustrative and not limiting in scope.
A. A non-aggregating protein analogue of a protein involved in a proteinopathy, the protein having a beta-sheet aggregation domain and the non-aggregating protein analogue comprising a beta-sheet destabilizing mutation in the beta-sheet aggregation domain, the non-aggregating protein analogue retaining substantially the wild type function of the protein.
B. A non-aggregating protein analogue as defined in the preceding aspect, comprising two or more beta-sheet destabilizing mutations in the beta-sheet aggregation domain.
C. A non-aggregating protein analogue as defined in any one of the preceding aspects, wherein the beta-sheet destabilizing mutation comprises substitution of a naturally occurring amino acid residue of the protein involved in the proteinopathy with a charged or polar amino acid residue, a glycine residue, a proline residue, or a beta-sheet destabilizing analogue of an amino acid.
D. A non-aggregating protein analogue as defined in the preceding aspect, wherein the charged or polar amino acid residue or analogue thereof comprises K, R, H, E, D, S, T, N, Q, C, or with an amino acid analogue that interferes with beta-sheet formation within the beta-sheet aggregation domain, wherein said amino acid analogues that interfere with beta-sheet formation are optionally 3-hydroxyproline, 4-hydroxyproline, selenocysteine, pyroglutamic acid, carboxyglutamic acid, octenyl alanine, pyrrolysine, palmitoyl aspartate, D-amino acids including D-proline, β-amino acids, γ-amino acids, Homo-amino acids, β-Homo-amino acids, α-methyl amino acids, N-methyl amino acids, N-ethyl amino acids, N-alkylated amino acid derivatives (preferably with 1, 2 or 3 carbons in the alkyl moiety), pyruvic acid derivatives, branched-chain amino acid derivatives, nitro amino acid derivatives, halogenated amino acid derivatives, ring-substituted amino acid derivatives, aromatic amino acid derivatives, linear core amino acids, peptoid derivatives, hydroxylated amino acid derivatives, cyclic amino acids, bicyclic amino acids, 3-amino-3-aryl-propionic acids, 3-amino-4-aryl-butyric acids, amino acids with aromatic spacers, alicyclic amino acids, or α-phenylglycine derivatives.
E. A non-aggregating protein analogue as defined in any one of the preceding aspects, wherein the beta-sheet destabilizing mutation comprises deletion of one or more naturally occurring amino acid residues of the protein involved in the proteinopathy, optionally including the deletion of up to or including all of the naturally occurring amino acid residues of the beta-sheet aggregation domain.
F. A non-aggregating protein analogue as defined in any one of the preceding aspects, wherein the beta-sheet destabilizing mutation comprises insertion of between one and five amino acid residues.
G. A non-aggregating protein analogue as defined in the preceding claim, wherein at least one of the inserted amino acid residues comprises K, R, H, E, D, S, T, N, Q, C, P or G, or an amino acid analogue that interferes with beta-sheet formation.
H. A non-aggregating protein analogue as defined in any one of the preceding aspects, wherein the protein involved in the proteinopathy comprises amyloid precursor protein (APP), amyloid beta protein (including Aβ-39, Aβ-40, Aβ-42 or Aβ-43 isoforms), alpha-synuclein, prion protein, Huntingtin protein, p53, or any of the proteins or peptides listed in Table 1.
I. A non-aggregating protein analogue as defined in any one of the preceding aspects, wherein the protein involved in the proteinopathy comprises amyloid beta, wherein optionally:
   the beta-sheet aggregation domain comprises positions 29-42 of the amyloid beta;
   the beta-sheet destabilizing mutation comprises a deletion or substitution of one of the following amino acid residues for the naturally occurring amino acid residue: G, P, K, R, H, E, D, S, T, N, Q, C, or an amino acid analogue that interferes with beta-sheet formation;
   the beta-sheet destabilizing mutation comprises an insertion of between one and five amino acid residues adjacent to the naturally occurring amino acid, optionally wherein at least one of the inserted amino acid residues comprises G, P, K, R, H, E, D, S, T, N, Q, C, or an amino acid analogue that interferes with beta-sheet formation;
   the non-aggregating protein analogue comprises a protein having the amino acid sequence set forth in any one of SEQ ID NO:2-193; and/or
   the non-aggregating protein analogue comprises a protein having the amino acid sequence set forth in any one of SEQ ID NOs: 194-207 or SEQ ID NOs: 712-742, wherein the X denotes a deletion, insertion of between one and five amino acid residues, optionally G, P, K, R, H, E, D, S, T, N, Q, C, or an amino acid analogue that interferes with beta-sheet formation, or substitution of one of the following amino acid residues for the naturally occurring amino acid: G, P, K, R, H, E, D, S, T, N, Q, C, or an amino acid analogue that interferes with beta-sheet formation.
J. A non-aggregating protein analogue as defined in any one of the preceding aspects, wherein the protein involved in the proteinopathy comprises alpha-synuclein, wherein optionally:
   the beta-sheet aggregation domain comprises positions 61-95 of the alpha-synuclein;
   the beta-sheet destabilizing mutation comprises a deletion or substitution of one of the following amino acid residues for the naturally occurring amino acid residue: G, P, K, R, H, E, D, S, T, N, Q, C, or an amino acid analogue that interferes with beta-sheet formation;
   the beta-sheet destabilizing mutation comprises an insertion of between one and five amino acid residues adjacent to the naturally occurring amino acid, optionally wherein at least one of the inserted amino acid residues comprises G, P, K, R, H, E, D, S, T, N, Q, C, or an amino acid analogue that interferes with beta-sheet formation;
   the non-aggregating protein analogue comprises a protein having the amino acid sequence set forth in any one of SEQ ID NO:209-698, wherein the X denotes a deletion, insertion of between one and five amino acid residues, optionally G, P, K, R, H, E, D, S, T, N, Q, C, or an amino acid analogue that interferes with beta-sheet formation, or substitution of one of the following amino acid residues for the naturally occurring amino acid: G, P, K, R, H, E, D, S, T, N, Q, C, or an amino acid analogue that interferes with beta-sheet formation.
K. A non-aggregating protein analogue as defined in any one of the preceding aspects, wherein the protein involved in the proteinopathy comprises prion protein, wherein optionally:
   the beta-sheet aggregation domain comprises positions 109-121 of the prion protein;
   the beta-sheet destabilizing mutation comprises a deletion or substitution of one of the following amino acid residues for the naturally occurring amino acid residue: G, P, K, R, H, E, D, S, T, N, Q, C, or an amino acid analogue that interferes with beta-sheet formation;
   the beta-sheet destabilizing mutation comprises an insertion of between one and five amino acid residues adjacent to the naturally occurring amino acid, optionally wherein at least one of the inserted amino acid residues comprises G, P, K, R, H, E, D, S, T, N, Q, C, or an amino acid analogue that interferes with beta-sheet formation; and/or
   the non-aggregating protein analogue comprises a protein having the amino acid sequence set forth in any one of SEQ ID NO:700-710, wherein the X denotes a deletion, insertion of between one and five amino acid residues, optionally G, P, K, R, H, E, D, S, T, N, Q, C, or an amino acid analogue that interferes with beta-sheet formation, or substitution of one of the following amino acid residues for the naturally occurring amino acid: G, P, K, R, H, E, D, S, T, N, Q, C, or an amino acid analogue that interferes with beta-sheet formation.
L. A nucleic acid encoding the non-aggregating protein analogue as defined in any one of the preceding aspects.
M. A nucleic acid as defined in the preceding aspect, comprising mRNA.
N. A liposome-based particle comprising a nucleic acid as defined in any one of the preceding aspects, the liposome-based particle being designed for delivery of the nucleic acid to a target cell to cause expression of the non-aggregating protein analogue.
O. A method for treatment and/or prophylaxis of a proteinopathy comprising administering to a subject having or at risk of developing the proteinopathy a peptide or nucleic acid as defined in any one of the preceding aspects.
P. A method as defined in the preceding claim, wherein the proteinopathy comprises Alzheimer's disease, Parkinson's disease, Lewy body disease or Lewy body dementia, Pick's disease, Creutzfeld-Jakob disease, Huntington's disease, amyotrophic lateral sclerosis (ALS), Down syndrome, neuronal degeneration with brain iron accumulation type I (Hallervorde-Spatz disease), Kuru or other transmissible spongiform encephalopathy (TSE), mild cognitive impairment (MCI), cerebral amyloid angiopathy (CAA), vascular dementias, or any neurodegenerative disease characterized by abnormal amyloid deposition, or any of the diseases or disorders listed in Table 1.
Q. A method as defined in any one of the preceding aspects, wherein administration is by intravenous, intracerebroventricular, intracerebral, intrathecal, intraperitoneal, intramuscular or intravenous injection, infusion, or topical, nasal, oral (including sublingual or buccal), anal, ocular or otic delivery; by liposome-mediated delivery including by direct delivery of the non-aggregating protein analogue as defined in any one of the preceding claims, direct delivery of a mRNA encoding the non-aggregating protein analogue as defined in any one of the preceding claims, or delivery of DNA encoding the non-aggregating protein analogue as defined in any one of the preceding claims together with suitable mechanisms (e.g. CRISPR-Cas gene editing systems) to integrate the DNA into the genome of the cell to facilitate expression of the non-aggregating protein analogue by the cell.
R. A method of designing a non-aggregating analogue of a protein implicated in a proteinopathy comprising the steps of:
   selecting the protein implicated in the proteinopathy;
   identifying a beta-sheet aggregation domain of the protein implicated in the proteinopathy; and
   identifying one or more beta-sheet destabilizing mutations within the beta-sheet aggregation domain.
S. A method as defined in the preceding aspect, wherein the step of identifying a beta-sheet aggregation domain of the protein implicated in the proteinopathy comprises conducting structural biology studies.
T. A method as defined in either one of the preceding aspects, wherein the step of identifying one or more beta-sheet destabilizing mutations within the beta-sheet aggregation domain comprises one or more of:
   deleting a naturally occurring amino acid residue from the beta-sheet aggregation domain and/or;
   replacing a naturally occurring amino acid residue in the beta-sheet aggregation domain with an amino acid residue that impairs formation of a cross-beta sheet structure.
U. A method as defined in the preceding aspect, wherein replacing the naturally occurring amino acid reside in the beta-sheet aggregation domain with an amino acid residue that impairs formation of a cross-beta sheet structure comprises replacing the naturally occurring amino acid residue with one of G, P, K, R, H, E, D, S, T, N, Q, or C, with an amino acid analogue that interferes with beta-sheet formation.
V. A non-aggregating protein analogue or method as defined in any one of the preceding aspects, wherein said amino acid analogues that interfere with beta-sheet formation are optionally 3-hydroxyproline, 4-hydroxyproline, selenocysteine, pyroglutamic acid, carboxyglutamic acid, octenyl alanine, pyrrolysine, palmitoyl aspartate, D-amino acids including D-proline, β-amino acids, γ-amino acids, Homo-amino acids, β-Homo-amino acids, α-methyl amino acids, N-methyl amino acids, N-ethyl amino acids, N-alkylated amino acid derivatives (preferably with 1, 2 or 3 carbons in the alkyl moiety), pyruvic acid derivatives, branched-chain amino acid derivatives, nitro amino acid derivatives, halogenated amino acid derivatives, ring-substituted amino acid derivatives, aromatic amino acid derivatives, linear core amino acids, peptoid derivatives, hydroxylated amino acid derivatives, cyclic amino acids, bicyclic amino acids, 3-amino-3-aryl-propionic acids, 3-amino-4-aryl-butyric acids, amino acids with aromatic spacers, alicyclic amino acids, or α-phenylglycine derivatives.

1-38. (canceled)

39. A pharmaceutical composition for treatment of Alzheimer's Disease, comprising a non-aggregating peptide analogue of the Aβ42 peptide, the Aβ42 peptide having an N-terminal domain corresponding to positions 1-28 of SEQ ID NO: 1 and a beta-sheet aggregation domain corresponding to positions 29-42 of SEQ ID NO: 1,
wherein the N-terminal domain of the analogue comprises an amino-acid sequence differing from residues 1-28 of SEQ ID NO: 1 by no more than 3 (preferably 2, more preferably 1, most preferably 0) deletions, insertions and/or substitutions, preferably conservative substitutions;
wherein the beta-sheet aggregation domain of the analogue comprises an amino-acid sequence differing from residues 29-42 of SEQ ID NO: 1 by no more than 5 deletions, insertions and/or substitutions; and
the non-aggregating peptide analogue substantially retaining native function of the Aβ42 peptide except for being non-aggregating.

40. The pharmaceutical composition of claim 39, wherein the retained native peptide function comprises capability of enhancing or inducing a7 nicotinic acetylcholine receptor (a7nAChR) mediated Ca²⁺ influx.

41. The pharmaceutical composition of claim 40, wherein the retained native peptide function is the capability of enhancing or inducing a7nAChR-mediated Ca²⁺ influx in cortical neurons at <3000 pM concentration, at <300 pM, at <30 pM, or at <3 pM.

42. The pharmaceutical composition of claim 39, wherein the beta-sheet aggregation domain of the analogue comprises an amino-acid sequence differing from residues 29-42 of SEQ ID NO: 1 by no more than 5 deletions, insertions and/or substitutions, preferably conservative substitutions.

43. The pharmaceutical composition of claim 39, wherein the beta-sheet destabilizing modification comprises substitution of a naturally occurring amino acid residue of the peptide with an amino acid analogue.

44. The pharmaceutical composition of claim 39, wherein the beta-sheet destabilizing modification comprises substitution of at most three naturally occurring amino acid residues at three separate positions relative to SEQ ID NO: 1 with an amino acid analogue.

45. The pharmaceutical composition of claim 39, wherein the amino acid analogue is one of: 3-hydroxyproline, 4-hydroxyproline, selenocysteine, pyroglutamic acid, carboxyglutamic acid, octenyl alanine, pyrrolysine, palmitoyl aspartate, D-amino acids, b-amino acids, y-amino acids, Homo-amino acids, b-Homo-amino acids, a-methyl amino acids, N-alkylated amino acid derivatives, pyruvic acid derivatives, branched-chain amino acid derivatives, nitro amino acid derivatives, halogenated amino acid derivatives, ring-substituted amino acid derivatives, aromatic amino acid derivatives, linear core amino acids, peptoid derivatives, hydroxylated amino acid derivatives, cyclic amino acids, bicyclic amino acids, 3-amino-3-aryl-propionic acids, 3-amino-4-aryl-butyric acids, amino acids with aromatic spacers, alicyclic amino acids, a-phenylglycine derivatives.

46. The pharmaceutical composition of claim 39, wherein the amino acid analogue comprises an N-alkylated amino acid analogue, preferably with 1-3 carbons in the alkyl moiety.

47. The pharmaceutical composition of claim 39, wherein the beta-sheet destabilizing modification comprises a substitution of M35, preferably with an amino acid analogue.

48. The pharmaceutical composition of claim 39, wherein the beta-sheet destabilizing modification comprises a substitution of M35 to proline, N-methyl methionine or pyrrolysine, preferably N-methyl methionine.

49. The pharmaceutical composition of claim 39, wherein the beta-sheet destabilizing modification comprises one or more of:
replacement of G29 with methyl glycine or N-ethyl glycine;
replacement of A30 with N-methyl alanine or N-ethyl alanine;
replacement of 131 is N-methyl isoleucine or N-ethyl isoleucine;
replacement of I32 with N-methyl isoleucine or N-ethyl isoleucine;
replacement of G33 with N-methyl glycine or N-ethyl glycine;
replacement of L34 with N-methyl leucine or N-ethyl leucine;
replacement of M35 with N-methyl methionine or N-ethyl methionine;
replacement of V36 with N-methyl valine or N-ethyl valine;
replacement of G37 with N-methyl glycine or N-ethyl glycine;
replacement of G38 with N-methyl glycine or N-ethyl glycine
replacement of V39 with N-methyl valine or N-ethyl valine;
replacement of V40 with N-methyl valine or N-ethyl valine;
replacement of 141 with N-methyl isoleucine or N-ethyl isoleucine; and/or
replacement of A42 with N-methyl alanine or N-ethyl alanine.

50. The pharmaceutical composition of claim 49, wherein the beta-sheet destabilizing modification comprises a replacement of M35 with N-methyl methionine or N-ethyl methionine.

51. The pharmaceutical composition of claim 39, having the amino acid sequence of one of SEQ ID NO:3, SEQ ID NO:88, SEQ ID NO: 100, SEQ ID NO: 190 or SEQ ID NO: 192 with at most three conservative amino acid substitutions in either or both of the N-terminal domain and the beta-sheet aggregation domain.

52. The pharmaceutical composition of claim 39, having the amino acid sequence of one of SEQ ID NO:88, SEQ ID NO:100, SEQ ID NO: 190 or SEQ ID NO:192.

53. The pharmaceutical composition of claim 39, wherein the pharmaceutical composition is a subcutaneous, intravenous, intracerebroventricular, intracerebral, intrathecal, intraperitoneal or intramuscular; topical, nasal, oral (including sublingual or buccal), rectal, ocular or otic pharmaceutical composition.

54. A method for treatment and/or prophylaxis of Alzheimer's disease comprising administering to a subject having or at risk of developing Alzheimer's disease a non-aggregating peptide analogue of the Aβ peptide, the Aβ peptide having an N-terminal domain corresponding to positions 1-28 of SEQ ID NO:1 and a beta-sheet aggregation domain corresponding to positions 29-42 of SEQ ID NO: 1
wherein the N-terminal domain of the analogue comprises an amino-acid sequence differing from residues 1-28 of SEQ ID NO: 1 by no more than 3 (preferably 2, more preferably 1, most preferably 0) deletions, insertions and/or substitutions, preferably conservative substitutions, and
wherein the beta-sheet aggregation domain of the analogue comprises an amino-acid sequence differing from residues 29-42 of SEQ ID NO: 1 by no more than 5 (preferably 3, more preferably 1, most preferably 0) deletions, insertions and/or substitutions.

55. The method of claim 54, wherein the non-aggregating peptide anal SEQ ID NO: 16 to SEQ ID NO:193.

56. The method of claim 54, wherein the method further comprises:
determining a concentration of Aβ42 in a sample of cerebrospinal fluid of the subject;
and
confirming that the measured concentration of Aβ42 in the cerebrospinal fluid sample is less than about 500 pM, preferably less than about 400 pM, preferably less than about 300 pM, more preferably less than 200 pM, identifying the subject as a candidate for treatment and/or prophylaxis of Alzheimer's disease prior to the administration of the non-aggregating peptide analogue of the Aβ peptide.
